(19) United States
(12) Patent Application Publication
(10) Pub. No.: US 2005/0025742 A1
(43) Pub. Date: Feb. 3, 2005

(54) Methods and compositions for interferon therapy

(75) Inventors: Heidrun Engler, San Diego, CA (US); Tattanahalli L. Nagabhushan, Parsippany, NJ (US); Stephen Youngster, Piscataway, NJ (US); Robert Connor, Oceanside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Canji, Inc., San Diego, CA (US)

(21) Appl. No.: 10/861,654

(22) Filed: Jun. 4, 2004

Related U.S. Application Data
(63) Continuation-in-part of application No. 10/455,215, filed on Jun. 4, 2003.
Application No. 10/455,215 is a continuation-in-part of application No. 10/055,863, filed on Jan. 22, 2002.
Application No. 10/055,863 is a continuation of application No. 09/112,074, filed on Jul. 8, 1998, now Pat. No. 6,392,069.
Application No. 09/112,074 is a continuation-in-part of application No. 08/889,355, filed on Jul. 8, 1997.
Application No. 08/889,355 is a continuation-in-part of application No. 08/584,077, filed on Jan. 8, 1996, now Pat. No. 5,789,244.
(63) Continuation-in-part of application No. 10/454,662, filed on Jun. 3, 2003.
Application No. 10/454,662 is a continuation of application No. 09/650,359, filed on Aug. 28, 2000, now abandoned.
Application No. 09/650,359 is a continuation of application No. 08/779,627, filed on Jan. 7, 1997, now Pat. No. 6,165,779.
Application No. 08/779,627 is a continuation-in-part of application No. 08/584,077, filed on Jan. 8, 1996, now Pat. No. 5,789,244.
(60) Provisional application No. 60/475,926, filed on Jun. 4, 2003.

(51) Int. Cl. A61K 38/21; A61K 48/00

(52) U.S. Cl. 424/85.4; 514/44; 424/85.7

(57) ABSTRACT

Methods and pharmaceutical compositions for administering protein or gene therapy to tissues or organs having an epithelial cell layer are provided. A protein or nucleic acid encoding the protein is administered to the target tissue or organ in combination with treatment with a delivery enhancing agent which increases the delivery of the interferon or nucleic acid to the cells of the target tissues or organs. The methods and combinations are particularly useful in the treatment of cancers and other conditions responsive to interferon therapy. An exemplary method comprises the transurethral intravesical administration to the bladder of a therapeutically effective amount of a pharmaceutical composition comprising an alpha-interferon or a gene delivery system encoding the interferon and SYN3 or a SYN3 homolog or analog. In the urinary bladder, as much as a 10 to 1000 fold increased in interferon levels and activity may be observed with the use of a SYN3 formulation as opposed to a PBS formulation of the same interferon protein or interferon gene delivery system.

### CROSS-REFERENCES TO RELATED APPLICATIONS

This application is a continuation-in-part of U.S. patent application Ser. No. 10/455,215 filed Jun. 4, 2003 which is a continuation-in-part of U.S. patent application Ser. No. 10/055,863, filed Jan. 22, 2002, which is a continuation of U.S. patent application Ser. No. 09/112,074, filed on Jul. 8, 1998 (U.S. Pat. No. 6,392,069, issued on May 21, 2002), which is a continuation in part of U.S. patent application Ser. No. 08/889,355, filed on Jul. 8, 1997, which is a continuation in part of U.S. patent application Ser. No. 08/584,077, filed Jan. 8, 1996 (U.S. Pat. No. 5,789,244, issued on Aug. 4, 1998); this application is also a continuation in part of U.S. patent application Ser. No. to be assigned, filed on Jun. 3, 2003, (Townsend and Townsend and Crew LLP Attorney Docket No. 016930-000815), which is a continuation of U.S. patent application Ser. No. 09/650,359, filed on Aug. 28, 2000, which is a continuation of U.S. patent application Ser. No. 08/779,627, filed Jan. 7, 1997 (U.S. Pat. No. 6,165,779, issued on Dec. 26, 2000), which is a continuation in part of U.S. patent application Ser. No. 08/584,077, filed on Jan. 8, 1996; this application claims priority to U.S. patent application Ser. No. to be assigned, filed on Jun. 4, 2004, (Townsend and Townsend and Crew LLP Attorney Docket No. 016930-000831US which claims the benefit of U.S. patent application No. 60/475926 filed on Jun. 4, 2003. This application contains subject matter related to that of U.S. patent application Ser. No. 10/329,043, filed on Dec. 20, 2002 which claims the benefit of U.S. patent application Ser. No. 60/342329 filed on Dec. 20, 2001. The disclosures of these priority applications are herein incorporated by reference in their entireties for all purposes.

### BACKGROUND OF THE INVENTION

Over 45,000 cases of superficial bladder cancer are diagnosed annually in the US (Cancer Facts and FIGS. 2002). Superficial disease is typically restricted to the surface mucosa (Ta) or present as carcinoma in situ (CIS), a flat surface-spreading variant. While some types of disease can be initially removed through transurethral resection of the bladder tumor (TURBT), the tendency for new tumor formation still remains high, possibly because macroscopic removal of the visible tumor may leave behind microscopic foci that will eventually result in tumor recurrence. In addition, surgical intervention is not possible for treatment of carcinoma in situ (CIS). Therefore, intravesical therapies have been developed as an adjuvant to surgery to prevent against tumor recurrence, or to eliminate small residual disease and/or inaccessible disease such as CIS.

Two general types of intravesical therapies are currently employed for the treatment of superficial bladder cancer: chemotherapy and BCG immunotherapy. Only three chemotherapeutic agents have shown efficacy via intravesical administration: thiotepa, adriamycin (and its derivatives epirubicin and valrubicin), and mitomycin. Multiple randomized trials comparing these chemotherapeutic agents to TURBT alone have revealed a net reduction in tumor recurrence of 12-15% (i.e., 60% recurrence with TURBT vs. 45% with TURBT plus chemotherapy) with no clear superiority of one agent over another (Traynelis et al., “Current status of intravesical therapy for bladder cancer In: S. N. Rous (ed.) Urology Annual,” Vol. 8, pp. 113-143, New York: WW Norton and Co, 1994). More importantly, in spite of this statistically significant reduction in recurrence, no evidence has been presented that demonstrates that chemotherapy reduces the chance of ultimate disease progression or improves survival (Lamm et al., J. Urol, 153:1444-50 (1995)).

Immunotherapy typically consists of intravesicular administration of the live vaccine strain of Mycobacterium bovis Bacillus Calmette-Guerin (“BCG”). Tumor recurrence is reduced about 30% using BCG following TURBT (or approximately twice that of intravesical chemotherapy) (O'Donnell M A, “Use of Intravesical BCG in Treatment of Superficial Bladder Cancer,” In: Droller, ed. Bladder Cancer: Current Diagnosis and Treatment, Totowa N.J.: Humana Press Inc., 2001). Ablation of small papillary tumors (<2 cm) can be achieved 55-60% of the time, and up to 75% for CIS (Kavoussi et al., J. Urol., 139:935 (1988)). Intravesical BCG has become the standard of care for patients with superficial bladder cancer who are at high-risk for recurrence or disease progression. In this regard, intravesical therapy with BCG can provide some degree of disease control and retention of a functional bladder, the desired goals for every patient with superficial bladder cancer. However, despite this BCG-mediated benefit for the initial treatment of high-risk superficial bladder cancer, about 50% of patients will relapse within 2 years. Commonly, such recurrences are retreated with BCG. However, it has been observed that patients treated with multiple intravesical BCG instillations may experience dose-limiting toxicity such as cystitis, dysuria, fever, and occasionally BCG sepsis. Accordingly, well-tolerated and effective alternatives to BCG treatment are desirable.

Recently investigators have evaluated intravesical recombinant interferon α2b protein (IntronA®) therapy for the treatment of superficial bladder cancer. Phase II human clinical studies demonstrated that intravesically instilled IntronA as a single agent at doses of 50-100 MIU resulted in a complete response in 40% of patients with superficial bladder cancer. (O'Donnell, et al., J. Urol., 2001 Oct., 166(4):1300-5).

In view of the above, it would be advantageous to have a method of improving the delivery of therapeutic proteins, polypeptides (e.g., interferons, Intron A) or gene delivery systems or nucleic acids to epithelial cells, and particularly, the urothelium.

### BRIEF SUMMARY OF THE INVENTION

In one of aspect of the invention, methods and pharmaceutical compositions for administering protein therapy to tissues or organs having an epithelial cell layer are provided. In one aspect, the protein therapy is administered to the target tissue or organ in combination with treatment with a protein therapy delivery enhancing agent which increases the transduction of the cells of the target tissues or organs by the vector. The methods and combinations can be useful in the treatment of cancers and other conditions responsive to therapy with a biologically active protein. In preferred embodiments, the delivery enhancing agent is SYN3.

In some embodiments, the protein therapy provides an interferon protein by administration of the interferon protein itself or by administration of an interferon gene delivery system wherein the gene encodes an interferon protein to be expressed in a transduced epithelial or urothelial cell. In an exemplary embodiment, the method comprises the transurethral intravesical administration to the bladder of a therapeutically effective amount of a pharmaceutical composition comprising SYN3 or a SYN3 homolog or analog and an interferon or an adenoviral vector encoding the interferon. In a further embodiment, the interferon is an α-interferon.

In another aspect, the present invention provides compositions and methods to enhance the delivery of nucleic acids to epithelial tissues. In one embodiment, the method provides contacting the cells of the tissue or organ with a gene delivery system wherein the gene encodes interferon in conjunction with delivery enhancing agent. In one further embodiment, the invention provides a pharmaceutical formulation comprising a recombinant adenovirus encoding an interferon gene and a delivery enhancing agent. In an exemplary embodiment, the delivery enhancing agent is SYN3 and the system comprises an adenovirus vector encoding a biologically active human interferon polypeptide.

In yet another aspect, the present invention provides compositions and methods to treat bladder cancer by enhancing the delivery of therapeutic proteins or gene delivery systems having a gene encoding the protein to the urinary bladder epithelium by intravesicular administration of a delivery enhancing agent and the therapeutic protein. In some embodiments, the therapeutic protein is an interferon. In further embodiments, the therapeutic protein is interferon α2b or interferon α2α1. In yet other embodiments, the delivery enhancing agent is SYN3 or a SYN3 homologue. In an exemplary embodiment, the delivery enhancing agent is SYN3 and the protein is an interferon (e.g., a biologically active human interferon polypeptide). In further embodiments, the protein may be administered as the protein or via a gene delivery system encoding the protein. In still other embodiments, the protein is an antibody or antibody fragment. In further embodiments, the antibody is directed toward a cytokine.

In another aspect, the invention provides a pharmaceutical composition for administration of a therapeutic protein and a delivery enhancing agent. In some embodiments, the protein is an interferon and the delivery enhancing agent is SYN3 or a SYN3 homologue. In an exemplary embodiment, the delivery enhancing agent is SYN3 and the protein is a biologically active human interferon polypeptide. In further embodiments, the interferon is interferon α2b or interferon α2α1.

In some aspects, the methods of the invention include treating any organ or tissue defining an interior space, sinus, ventricle, passage, volume, cavity, void or lumen lined with or containing the epithelial membrane by intravesicular administration of a delivery enhancing agent and a therapeutic protein or a nucleic acid encoding a therapeutic protein. The surfaces or walls defining such serve to contain or retard or limit the bulk fluid movement or transfer of the pharmaceutical composition to another body portion and can allow a longer contact time of the epithelial membrane with the pharmaceutical composition. The organ or tissue can be a bladder (e.g., the urinary bladder) which has an epithelial membrane at the inner surface. In some embodiments, the organ is the stomach, uterus, the intestine, the esophagus, the mouth, the colon, the upper or lower GI tract, or the upper or lower respiratory tract. In some embodiments, the organ or tissue defines a space such as the peritoneal cavity and the epithelial surface is located on an epithelial surface capable of making fluidic contact with the space within the peritoneal cavity. In some embodiments, the organ or tissue has cancer, a proliferative disorder or an infectious disease and the therapeutic protein is an interferon and the delivery enhancing agent is SYN3 or a SYN3 analog.

### BRIEF DESCRIPTION OF THE DRAWINGS

FIG. 1 provides a schematic representation of a replication deficient recombinant adenoviral gene delivery system used in the experiments described herein to demonstrate the effects of delivery enhancing agents to enhance gene expression in tumor cells.

FIG. 2 is a graphical representation depicting the antitumor efficacy of a pharmaceutical composition comprising a recombinant adenoviral interferon gene delivery system and SYN3 in a subcutaneous xenograft tumor models. Inhibition of tumor growth was observed following intratumoral rAd-IFN administration in nude mice bearing tumors derived from glioblastoma (LN229, U87MG), hepatoma (HEP3B) and CML (K562). Inhibition of subcutaneous xenograft tumor growth by intratumoral administration. Groups of 5 mice were treated with injections 3 days/week for 2 weeks of vehicle, rAd-control, or rAd-IFN (1×10¹⁰ particles/dose; total dose 6×10¹⁰ particles).

FIG. 3 shows the ability of SYN3 to enhance the delivery of a recombinant adenoviral gene delivery system in the rat. Female Sprague-Dawley rats received intravesical administration of rAd-β-gal (7.6×10¹⁰ P/ml) in either a SYN3 formulation (1 mg/ml in vehicle) or in vehicle alone (0.1% Tween-80) for 45 min. After 48h, animals were sacrificed, their bladders harvested, fixed and X-gal stained for lacZ expression.

FIG. 4 shows the effects of pharmaceutical composition comprising an interferon gene delivery system with SYN3 on tumors in a mouse urinary bladder tumor model. Female nude mice were catheterized trans-urethrally, and received 100 ml of trypsin-EDTA (0.25%) for 30 min prior to receiving UMUC-3 cells (1×107; 100 ml) for 3 hours. After 6d, the animals were dosed 2× (at 24 hour intervals; d6, d7)) via intravesical administration of either rAd-IFN or rAd-control (1×10¹¹/100 μl), or SYN3 orily (1 mg/ml). Twenty-one days after tumor cell implantation, the animals were sacrificed, their bladders harvested into formalin for macroscopic examination and scoring of tumor burden. Tumors were scored as follows: 0=no tumor, 1=<25% of bladder lumen occluded; 2=25-50% of bladder lumen occluded; 3=>50% of bladder lumen occluded.

FIG. 5 provides data relating to the efficacy of SYN3 in conjuction with a gene delivery enhancing system an orthotopic tumor model of superficial bladder cancer in which superficial tumors were established in the bladders of athymic mice by intravesical administration of human KU-7 bladder cancer cells stably transfected with the Green Fluorescent Protein (GFP) following trypsin pre-treatment. By surgically exposing and illuminating the bladder to activate GFP fluorescence, the sequential evaluation of tumor size can be monitored in vivo without sacrifice of the animal. Tumor cells were allowed to grow for a period of 10 days, whereupon the bladder was surgically exposed and illuminated, and the fluorescent tumor burden for each animal photographically recorded. Using image analysis software, the percentage area of the bladder containing tumor cells relative to the total area of the bladder was also determined before and after treatment. Mice received intravesical administration of 100 μl of rAd (1×10¹¹ P/ml) for one hour on two consecutive days. Four treatment groups were compared: rAd-IFN/SYN3, rAd-β-gall/SYN3 rAd-IFN alone or SYN3 alone. Twenty-one days after treatment (31d after initiation of tumor growth), the bladders were again inflated, surgically exposed and the GFP tumor burden was again measured as described above. Animals that received the rAd-IFN/SYN3 had a significant decrease in the tumor burden over time.

FIG. 6 shows the effects of a pharmaceutical composition comprising SYN3 and an interferon gene delivery system on the levels of interferon in blood, urine, and bladder tissue. Mice received intravesical administration (100 μl; 7.4×10¹⁰ P/ml) of either rAd-IFN (IACB) or rAd-control (ZZCB) in a SYN3 formulation (1 mg/ml). Two days after treatment urine and serum was obtained from each animal, whereupon animals were sacrificed and their bladders harvested, homogenized and assayed for levels of IFN. This data provides that the level of interferon expression may be determined by assay of urine and that there is no discemable systemic exposure of the animal to interferon protein following intravesical administration of the gene delivery system encoding interferon.

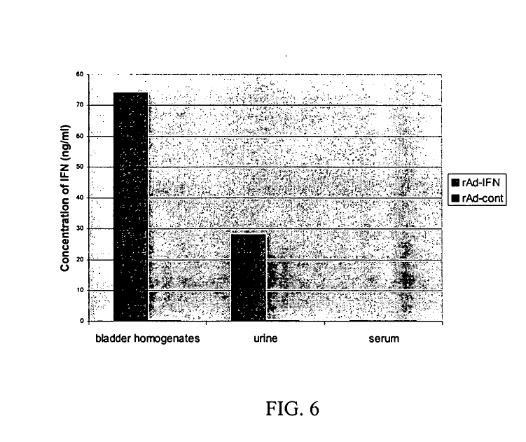

FIG. 7 illustrates one method for the synthesis of SYN3.

FIG. 8 shows the effect of a SYN3 formulation as opposed to PBS on the uptake of interferon by urothelium in the rat at various time points. As the supply of the hybrid IFN protein is limited, the IFNα2b protein (Intron A) was primarily used. For comparison purposes, the hybrid protein was also be included at the time t=0 time point.

FIG. 9 shows the effect of SYN3 formulation as opposed to PBS on the interferon expression of: 2′,5′-oligoadenylate synthetase (OAS); in the rat.

FIG. 10 shows the effect of SYN3 formulation as opposed to PBS on the interferon induced expression of: MX1 in the rat.

FIG. 11 shows the effect of SYN3 formulation as opposed to PBS on any interferon induced expression of: IRF1 in the rat.

FIG. 12 shows the effect of SYN3 formulation as opposed to PBS on the interferon expression of: INFγ in the rat.

### DETAILED DESCRIPTION OF THE INVENTION

Unless otherwise defined, all technical and scientific terms used herein have the same meaning as commonly understood by one of ordinary skill in the art to which this invention belongs. Each publication, patent application, patent, and other reference cited herein is incorporated by reference in its entirety to the extent that it is not inconsistent with the present disclosure.

As used in this specification and the appended claims, the singular forms “a,” “tan,” and “the” include plural reference unless the context clearly dictates otherwise.

In one aspect, the present invention provides compositions and methods to enhance the delivery of proteins or nucleic acids to tissues having an epithelial layer or urothelium. In one embodiment, the method provides contacting the cells of the tissue or organ with an interferon, or a gene delivery system encoding an interferon, in conjunction with delivery enhancing agent.

In some embodiments, the present invention provides compositions and methods to enhance the delivery of proteins (e.g., an interferon) to epithelium by contacting the epithelium with a delivery enhancing agent (e.g., SYN3). In one embodiment, the method provides contacting the urothelium of the bladder with 1) interferon or an interferon gene delivery system and 2) a SYN3 or a SYN3 homolog. The invention relates to the discovery that SYN3 enhanced delivery of gene delivery systems and proteins to the bladder is a highly effective means of delivering protein (e.g., interferon) biological activity in amounts rivaling those obtained with recombinant adenoviral gene therapy using SYN3. These results indicate in particular that SYN3 enhanced delivery of interferon will be useful in treating bladder cancer. The results also indicate that in general, SYN3 and its homologs should also be effective in delivering other therapeutic proteins across epithelial barriers to the cells of an epithelial tissue, particularly with respect to the urothelium.

Gene Delivery System:

A “gene delivery system” comprises a recombinant polynucleotide encoding a biologically active protein operatively linked to expression control sequence to effect expression of the protein gene in a cell. An interferon gene delivery system comprises a recombinant polynucleotide encoding an interferon operatively linked to expression control sequences to effect expression of the interferon gene in a cell.

The term polynucleotide refers to a polymer composed of nucleotide monomers. Polynucleotides include naturally the occurring nucleic acids, such as deoxyribonucleic acid (“DNA”) and ribonucleic acid (“RNA”) as well as nucleic acid analogs. Nucleic acid analogs include those non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidites, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally less than about 50 nucleotides. It will be understood that when a nucleotide sequence is represented by a DNA sequence (i.e., A, T, G, C), this also includes an RNA sequence (i.e., A, U, G, C) in which “U” replaces “T.” “Recombinant polynucleotide” refers to a polynucleotide having sequences that are not naturally joined together. An amplified or assembled recombinant polynucleotide may be included in a suitable vector, and the vector can be used to transform a suitable host cell of the subject. A host cell that comprises the recombinant polynucleotide is referred to as a “recombinant host cell.” The gene is then expressed in the recombinant host cell to produce, e.g., a “recombinant interferon polypeptide.”

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an interferon amino acid sequence or polypeptide” includes all nucleotide sequences that are degenerate versions of each other and that encode the same amino acid sequence. Nucleotide sequences that encode proteins and RNA may include introns.

“Expression control sequence” refers to a nucleotide sequence in a polynucleotide that regulates the expression (transcription and/or translation) of a nucleotide sequence operatively linked thereto. “Operatively linked” refers to a functional relationship between two parts in which the activity of one part (e.g., the ability to regulate transcription) results in an action on the other part (e.g., transcription of the sequence). Expression control sequences can include, for example and without limitation, sequences of promoters (e.g., inducible or constitutive), enhancers, transcription terminators, a start codon (i.e., ATG), splicing signals for introns, and stop codons.

An expression vector comprises sufficient cis-acting elements for expression; other elements for expression can be supplied by the host cell expression system. Expression vectors include all those known in the art, such as cosmids, plasmids (e.g., naked or contained in liposomes) and viruses that incorporate the recombinant polynucleotide.

In one embodiment, a particular expression control sequence may employed to provide selective expression of the interferon gene in a particular type of tissue by the use of a promoter and/or other expression elements preferentially used by the tissue of interest. Examples of known tissue-specific promoters include the promoter for creatine kinase, which has been used to direct the expression of dystrophin cDNA expression in muscle and cardiac tissue (Cox et al., Nature, 364:725-729 (1993)); immunoglobulin heavy or light chain promoters for the expression of genes in B cells; albumin or alpha-fetoprotein promoters to target cells of liver lineage and hepatoma cells, respectively. Exemplary tissue-specific expression elements for the liver include, but are not limited, to HMG-CoA reductase promoter (Luskey, Mol. Cell. Biol., 7(5):1881-1893 (1987)); sterol regulatory element 1 (SRE-1; Smith et al., J Biol. Chem., 265(4):2306-2310 (1990); phosphoenol pyruvate carboxy kinase (PEPCK) promoter (Eisenberger et al., Mol. Cell Biol., 12(3):1396-1403 (1992)); human C-reactive protein (CRP) promoter (Li et al., J. Biol. Chem., 265(7):4136-4142 (1990)); human glucokinase promoter (Tanizawa et al., Mol. Endocrinology, 6(7):1070-81 (1992); cholesterol 7-alpha hydroylase (CYP-7) promoter (Lee et al., J. Biol. Chem., 269(20):14681-9 (1994)); beta-galactosidase alpha-2,6 sialyltransferase promoter (Svensson et al., J. Biol. Chem., 265(34):20863-8 (1990); insulin-like growth factor binding protein (IGFBP-1) promoter (Babajko et al., Biochem Biophys. Res. Comm., 196 (1):480-6 (1993)); aldolase B promoter (Bingle et al., Biochem J., 294(Pt2):473-9 (1993)); human transferrin promoter (Mendelzon et al., Nucl. Acids Res., 18(19):5717-21 (1990); collagen type I promoter (Houglum et al., J. Clin. Invest., 94(2):808-14 (1994)). Exemplary tissue-specific expression elements for the prostate include, but are not limited to, the prostatic acid phosphatase (PAP) promoter (Banas et al., Biochim. Biophys. Acta., 1217(2):188-94 (1994); prostatic secretory protein of 94 (PSP 94) promoter (Nolet et al., Biochim. Biophys. ACTA, 1098(2):247-9 (1991)); prostate specific antigen complex promoter (Casper et al., J. Steroid Biochem. Mol. Biol., 47 (1-6): 127-35 (1993)); human glandular kallikrein gene promoter (hgt-1) (Lilja et al., World J. Urology, 11(4):188-91 (1993). Exemplary tissue-specific expression elements for gastric tissue include, but are not limited to, the human H⁺/K⁺-ATPase alpha subunit promoter (Tanura et al., FEBS Letters, 298:(2-3):137-41 (1992)). Exemplary tissue-specific expression elements for the pancreas include, but are not limited to, pancreatitis associated protein promoter (PAP) (Dusetti et al., J. Biol. Chem., 268(19):14470-5 (1993)); elastase 1 transcriptional enhancer (Kruse et al., Genes and Development, 7(5):774-86 (1993)); pancreas specific amylase and elastase enhancer promoter (Wu et al., Mol. Cell. Biol., 11(9):4423-30 (1991); Keller et al., Genes & Dev., 4(8):1316-21 (1990)); pancreatic cholesterol esterase gene promoter (Fontaine et al., Biochemistry, 30(28):7008-14 (1991)). Exemplary tissue-specific expression elements for the endometrium include, but are not limited to, the uteroglobin promoter (Helftenbein et al., Annal. NY Acad. Sci., 622:69-79 (1991)). Exemplary tissue-specific expression elements for adrenal cells include, but are not limited to, cholesterol side-chain cleavage (SCC) promoter (Rice et al., J. Biol. Chem., 265:11713-20 (1990). Exemplary tissue-specific expression elements for the general nervous system include, but are not limited to, gamma-gamma enolase (neuron-specific enolase, NSE) promoter (Forss-Petter et al., Neuron, 5(2):187-97 (1990)). Exemplary tissue-specific expression elements for the brain include, but are not limited to, the neurofilament heavy chain (NF-H) promoter (Schwartz et al., J. Biol. Chem., 269(18):13444-50 (1994)). Exemplary tissue-specific expression elements for lymphocytes include, but are not limited to, the human CGL-1/granzyme B promoter (Hanson et al., J. Biol. Chem., 266 (36):24433-8 (1991)); the terminal deoxy transferase (TdT), lambda 5, VpreB, and 1ck (lymphocyte specific tyrosine protein kinase p561ck) promoter (Lo et al., Mol. Cell. Biol., 11(10):5229-43 (1991)); the humans CD2 promoter and its 3′transcriptional enhancer (Lake et al., EMBO J., 9(10):3129-36 (1990)), and the human NK and T cell specific activation (NKG5) promoter (Houchins et al., Immunogenetics, 37(2):102-7 (1993)). Exemplary tissue-specific expression elements for the colon include, but are not limited to, pp60c-src tyrosine kinase promoter (Talamonti et al., J. Clin. Invest, 91(1):53-60 (1993)); organ-specific neoantigens (OSNs), mw 40kDa (p40) promoter (Ilantzis et al., Microbiol. Immunol., 37(2):119-28 (1993)); colon specific antigen-P promoter (Sharkey et al., Cancer, 73(3 supp.) 864-77 (1994)). Exemplary tissue-specific expression elements for breast cells include, but are not limited to, the human alpha-lactalbumin promoter (Thean et al., British J. Cancer., 61(5):773-5 (1990)).

The use of the term “interferon gene” refers to a gene that directs the expression of an interferon.

The term “gene” as used herein is intended to refer to a nucleic acid sequence which encodes an polypeptide. This definition includes various sequence polymorphisms, mutations, and/or sequence variants wherein such alterations do not affect the function of the gene product. The term “gene” may include not only coding sequences but also regulatory regions such as promoters, enhancers, and termination regions. The term further can include all introns and other DNA sequences spliced from the mRNA transcript, along with variants resulting from alternative splice sites. Nucleic acid sequences encoding the polypeptide can be DNA or RNA which directs the expression of a specific protein or peptide. These nucleic acid sequences may be a DNA strand sequence that is transcribed into RNA or an RNA sequence that is translated into protein. The nucleic acid sequences include both the full-length nucleic acid sequences as well as non-full length sequences derived from the full-length protein. It is further understood that the sequence includes the degenerate codons of the native sequence or sequences that may be introduced to provide codon preference in a specific host cell.

In one aspect, the present invention provides compositions and methods to enhance the delivery of proteins (e.g., an interferon) to epithelium by contacting the epithelium with a delivery enhancing agent (e.g., SYN3 or a SYN3 homolog). The protein can be a cytokine (e.g., interleukins, interferons, colony stimulating factors), an anti-metastatic factor, or tumor suppressor protein. In some embodiments, the protein is any one or more of macrophage colony stimulating factor (GMCSF), granulocyte colony stimulating factor (GCSF), macrophage colony stimulating factor (MCSF),interleukin-1 (IL-1), interleukin-2 (IL-2), interleukin-6 (IL-6), interleukin-9 (IL-9), interleukin-11 (IL-I1), interleukin-12 (IL-12), interleukin-13 (IL-13), interleukin-18 (IL-18), heat shock protein (HSP), p53, and an anti-angiogenic factor (protein agents that oppose angiogenic action of vascular endothelial cell growth factor (VEGF), anti-metastatic factors, e.g., tissue inhibitors of metalloproteinases (TIMPs), and factors that increase BCG activity.

In some embodiments, the protein of the protein therapy is an antibody which may be a monoclonal antibody or polyclonal antibody or a Fab fragment. The antibodies may be chimeric or humanized. These antibodies would include, but are not limited to, antibodies to cytokines and Type I and Type II interferons (e.g., anti-interferon-α, anti-interferon-β, anti-interferon-δ, anti-interferon-γ), and antibodies against the interleukins (e.g., anti-IL-1, anti-IL-2, anti-IL-4, anti-I1-6, anti-IL-7 and anti-IL-10). In the context of the present invention, antibodies are understood to include, for example, monoclonal antibodies, polyclonal antibodies, antibody fragments (e.g., Fab, and F(ab′)2) and recombinantly produced binding partners. Antibodies are understood to be reactive against the target if the Ka is greater than or equal to 10⁻⁷ M.

Polyclonal antibodies may be readily generated by one of ordinary skill in the art from a variety of warm-blooded animals. Monoclonal antibodies may also be readily generated using conventional techniques (see, e.g., U.S. Pat. Nos. RE 32,011, 4,902,614; 4,543,439; and 4,411,993; see, also, Kennett, McKeam, and Bechtol (eds.) Monoclonal Antibodies, Hybridomas: A New Dimension in Biological Analyses, Plenum Press, (1980); and Harlow and Lane (eds.) Antibodies: A Laboratory Manual, Cold Spring Harbor Laboratory Press (1988)). Preparation of preferred antibodies is further described in the examples section, below.

The term interferon as used herein is intended to include all classes and subclasses of interferon polypeptides, and deletion, insertion, or substitution variants, including conservative amino acid substitutions, thereof, biologically active polypeptide fragments thereof, and allelic forms thereof. A variety of suitable interferon polypeptides are known to those of ordinary skill in the art. In some embodiments, the interferon polypeptide is Type I or Type II interferon, including those commonly designated as alpha-interferon, beta-interferon, gamma-interferon, and omega-interferon (e.g., α-interferon, β-interferon, γ-interferon and \acute{ω}-interferon), and combinations thereof, including the consensus sequence for alpha-interferon. In some embodiments, the alpha-interferon is alpha₁, or alpha₂-interferon. In some embodiments, the gene encodes interferon α-2b.

In some embodiments, the interferon protein (as administered as a protein or encoded by the gene delivery system) is a wild-type interferon polypeptide. In other embodiments, the interferon is substantially identical to a wild-type interferon polypeptide or a fusion protein thereof. The terms “identical” or percent “identity,” in the context of two or more polynucleotide or polypeptide sequences, refer to two or more sequences or subsequences that are the same or have a specified percentage of nucleotides or amino acid residues that are the same, when compared and aligned for maximum correspondence. In the context of two nucleic acids or polypeptides, the term “substantially identical” refers to two or more sequences or subsequences that have at least 70% nucleotide or amino acid residue identity when compared and aligned for maximum correspondence, as measured using the BLAST algorithm, which is described in Altschul et al., J. Mol. Biol., 215:403-410 (1990). Software for performing BLAST analyses is publicly available through the National Center for Biotechnology Information (http://www.ncbi.nlm.nih.gov/). In addition to calculating percent sequence identity, the BLAST algorithm also performs a statistical analysis of the similarity between two sequences (see, e.g., Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

In other embodiments, the interferon polypeptide (as administered as a protein or encoded by the gene delivery system) is substantially identical to the sequence of a native or wild-type polypeptide over a stretch of 50 amino acids, 100 amino acids, 150 amino acids or the entire length of the native polypeptide. In some embodiments, the interferon polypeptide (to be administered or encoded by the gene delivery system) is 99%, 98%, 95% or 90% identical to the sequence of a wild-type interferon polypeptide over a stretch of 50 amino acids, 100 amino acids, or the entire length of the native polypeptide. In some embodiments, the interferon polypeptide (as administered as a protein or encoded by the gene delivery system) is substantially identical to the sequence of a human wild-type alpha-interferon polypeptide over a stretch of 50 amino acids, 100 amino acids, or the entire length of the native polypeptide. In some embodiments, the interferon polypeptide (as administered as a protein or encoded by the gene delivery system) encoded by the gene delivery system is 99%, 98%, 95% or 90% identical to the sequence of the human wild-type polypeptide over a stretch of 50 amino acids, 100 amino acids, 150 amino acids or the entire length of the native polypeptide.

In some embodiments, the interferon (as administered as a protein or encoded by the gene delivery system) is a hybrid interferon. The construction of hybrid alpha-interferon genes containing combinations of different interferon subtype sequences (e.g., α and Δ, α and β, and α and F) is disclosed in U.S. Pat. Nos. 4,414,150, 4,456,748, and 4,678,751. U.S. Pat. Nos. 4,695,623, 4,897,471 and 5,831,062 disclose novel human leukocyte interferon polypeptides having amino acid sequences which include common or predominant amino acids found at each position among naturally-occurring alpha interferon subtype polypeptides and are referred to as consensus human leukocyte interferon. In one embodiment of the invention, the hybrid interferon is interferon α2 al.

In some embodiments, the interferon (to be administered as a protein or encoded by the gene delivery system) is an interferon-α Recombinant interferon alphas, for instance, have been cloned and expressed in E. coli (e.g., Weissmann et al., Science, 209:1343-1349 (1980); Sreuli et al., Science, 209:1343-1347 (1980); Goeddel et al., Nature, 290:20-26 (1981); Henco et al., J. Mol. Biol., 185:227-260 (1985)). In some such embodiments, the interferon is a human interferon alpha. In some embodiments, the interferon alpha is interferon alpha 2a or 2b (see, for example, WO 91/18927). In some embodiments, the protein is an interferon with anti-cancer, anti-infective, or immune system modulating bioactivity. In some embodiments the administered interferon protein is a semisynthetic protein-polymer conjugate (e.g., a PEGylated interferon). In some exemplary embodiments, the interferon is a type I interferon-alpha (IFN-alpha) with anti-infective and antitumor activity or a PEGylated IFN-alpha2b. A 12,000-Da monomethoxypolyethylene glycol (PEG-12000) polymer can be attached. PEG conjugation is thought to increase the serum half-life and thereby prolong patient exposure to IFN-alpha2b without altering the biologic potency to the protein.

The term “polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. The term “protein” typically refers to large polypeptides. The term “peptide” typically refers to short polypeptides . “Conservative substitution” refers to the substitution in a polypeptide of an amino acid with a functionally or structurally similar amino acid. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

The term “biologically active” as used herein refers to any anti-viral or anti-proliferative or anti cancer activity as measured by techniques well known in the art (see, for example, Openakker et al., supra; Mossman, J. Immunol. Methods, 65:55 (1983)).

“Allelic form” refers to any of two or more polymorphic forms of a polypeptide occupying the same genetic locus. Allelic variations arise naturally through mutation, and may result in phenotypic polymorphism within populations. Gene mutations can be silent (no change in the encoded polypeptide) or may encode polypeptides having altered amino acid sequences. “Allelic forms” also refer to cDNAs polypeptides derived from MRNA transcripts of genetic allelic variants. In some embodiments, the interferon is an allelic form.

As used herein, the term “alkyl” denotes branched, unbranched, or cyclic hydrocarbon substituent or combination thereof, which may be fully saturated, mono- or polyunsaturated and can include di- and multivalent substituents, having the number of carbon atoms designated (i.e. C₁-C₁₀-means one to ten carbons) Examples of saturated hydrocarbon substituents include, but are not limited to, groups such as methyl, ethyl, n-propyl, iso-propyl, n-butyl, sec-butyl, iso-butyl, tert-butyl, octa-decyl, 2-methylpentyl, cyclohexyl, (cyclohexyl)methyl, cyclopentylmethyl. The substituents can be optionally substituted with one or more functional groups which are attached commonly to such chains, such as hydroxyl, bromo, fluoro, chloro, iodo, mercapto, or thio, cyano, alkylthio, aryl, heteroaryl, carboxyl, nitro, amino, alkoxyl, amido, and the like to form alkyl substituents such as carboxymethyl, trifluoromethyl, 3-hydroxyhexyl, 2-carboxypropyl, and the like. An unsaturated alkyl substituent is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The substituents can be substituted with one or more functional groups which are attached commonly to such chains as described for saturated hydrocarbons.

The term “aryl” means a polyunsaturated, typically aromatic, hydrocarbon substituent which can be a single ring or multiple rings (up to three rings) which are fused together or linked covalently.

The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. The above noted aryl and heteroaryl ring systems can be further substituted with one or more functional groups which are attached commonly to such ring systems such as hydroxyl, bromo, fluoro, chloro, iodo, mercapto, thio, cyano, alkylthio, carboxyl, nitro, amino, alkoxyl, or amido.

The term “acyl” denotes the —C(O)R— substituent, wherein R is alkyl or aryl as defined above, such as but not limited to benzoyl, succinyl, acetyl, propionyl or butyryl.

The term “hydroxyl” denotes the substituent —OH—.

The term “alkoxy” denotes the substituent —OR— where R is alkyl.

The term “amino” denotes an amine linkage (—NRR′) where R and R′ are independently hydrogen substituent, alkyl substituent, or aryl substituent.

The term “carboxylate” denotes the substituent —OC(O)R—, wherein R is an optionally substituted alkyl or aryl.

The term “acyloxy” denotes the substituent —(CRR′)ₘC(O)OR″—, wherein R and R′ are independently selected from a group comprising of an alkyl substituent, aryl substituent or hydrogen substituent and R″ is hydrogen or an alkyl substituent and m is an integer between 1-8, inclusive.

The term “halogen” or “halo” refers to the substituents F, Cl, Br, or I.

The term “saccharide residue” refers to a monosaccharide substituent which can include more than one monosaccharide substituent linked as a homo-oligosaccharide substituent (an oligosaccharide comprising one type of monosaccharide) or hetero-oligosaccharide substituent (an oligosaccharide comprising more than one type of monosaccharide). In a preferred embodiment, the homo and hetero-oligosaccharide substituent is composed of 2 to 10 monosaccharide units. Monosaccharides can include pentose or hexose residues and the residues can exist as the cyclized or uncyclized (open-chain) form. When a monosaccharide is in the open chain form, the oxygen atom of the carbonyl carbon can be replaced with —RR′— where R and R′ are independently selected from a group of an alkyl substituent, a halogen substituent, a hydroxyl substituent, a hydrogen substituent, a amino substituent, or a alkoxy substituent. Preferred oligo-saccharides include a pentose-pentose disaccharide group, a hexose-hexose disaccharide group, a pentose-hexose disaccharide group, and a hexose pentose disaccharide group. The monosaccharide can be selected from a group of ribose, arabinose, xylose and lyxose, allose, altrose, glucose, mannose, gulose, idose, galactose, or talose, whereby one or more the hydroxyl groups on the monosaccharide can be replaced with hydrogen, alkyl substituent, alkoxy substituent, amino substituent, or an acyl substituent.

The bladder epithelium or “urothelium,” lines the inner bladder surface and is thus proximal to the contents thereof. Urothelium also lines the inner surface of the renal pelvis, the ureters, and the urinary bladder, and forms a tight barrier that allows for retention of urine, while preventing the unregulated movement of ions, solutes, and toxic metabolites across the epithelial barrier. This permeability barrier must normally be maintained even as the organ undergoes cyclical changes in pressure as it fills and empties. Most bladder cancers originate from the cells of this epithelium. As the urethra, ureters and the pelvis of the kidney are also lined by this transitional epithelium, the same types of cancer seen in the bladder can also occur at these sites. In some embodiments, the methods and compositions can be used to treat such cancers at these sites in the urethra, ureters and the pelvis of the kidney.

The gene delivery system generally is provided in the form of a eucaryotic expression vector capable of directing the expression of the interferon gene in a eucaryotic cell. Examples of eucaryotic expression vectors include viral and non-viral vectors. The term “eucaryotic expression vector” refers to viral and non-viral vectors prepared by conventional recombinant DNA techniques comprising interferon expression cassette. The term “expression cassette” is used herein to define a nucleotide sequence capable of directing the transcription and translation of a interferon coding sequence comprising expression control elements operably linked to a interferon coding sequence so as to result in the transcription and translation of a the interferon sequence in a transduced mammalian cell. A variety of viral and non-viral delivery vectors useful to achieve expression of nucleotide sequences in transduced cells are known in the art. See, e.g. Boulikas, T in Gene Therapy and Molecular Biology, Volume 1 (Boulikas, T. Ed.) 1998 Gene Therapy Press, Palo Alto, Calif. pages 1-172

Examples of non-viral delivery systems used to introduce the interferon gene to a target cell include expression plasmids capable of directing the expression of the interferon. Expression plasmids are autonomously replicating, extrachromosomal circular DNA molecules, distinct from the normal genome and nonessential for cell survival under nonselective conditions capable of effecting the expression of a DNA sequence in the target cell. The expression plasmid may also contain promoter, enhancer or other sequences aiding expression of the therapeutic gene and/or secretion can also be included in the expression vector. Additional genes, such as those encoding drug resistance, can be included to allow selection or screening for the presence of the recombinant vector. Such additional genes can include, for example, genes encoding neomycin resistance, multi-drug resistance, thymidine kinase, beta-galactosidase, dihydrofolate reductase (DHFR), and chloramphenicol acetyl transferase.

The expression plasmid containing the interferon gene may be encapsulated in liposomes. Liposomes include emulsions, foams, micelles, insoluble monolayers, liquid crystals, phospholipid dispersions, lamellar layers and the like. The delivery of nucleic acids to cells using liposome carriers is well known in the art. A variety of methods are available for preparing liposomes, as described in, e.g., Szoka et al. Ann. Rev. Biophys. Bioeng. 9:467 (1980), Szoka, et al. U.S. Pat. No. 4,394,448 issued Jul. 19, 1983, as well as U.S. Pat. Nos. 4,235,871, 4,501,728, 4,837,028, and 5,019,369. Liposomes useful in the practice of the present invention may be formed from one or more standard vesicle-forming lipids, which generally include neutral and negatively charged phospholipids and a sterol, such as cholesterol. Examples of such vesicle forming lipids include DC-chol, DOGS, DOTMA, DOPE, DOSPA, DMRIE, DOPC, DOTAP, DORIE, DMRIE-HP, n-spermidine cholesterol carbamate and other cationic lipids as disclosed in U.S. Pat. No. 5,650,096. The selection of lipids is generally guided by consideration of, e.g., liposome size, acid lability and stability of the liposomes in the blood stream. Additional components may be added to the liposome formulation to increase serum half-life such as polyethylene glycol coating (so called “PEG-ylation”) as described in U.S. Pat. Nos. 5,013,556 issued May 7, 1991 and 5,213,804 issued May 25, 1993.

In order to provide directed delivery of the non-viral interferon gene delivery system to a particular cell, it may be advantageous to incorporate elements into the non-viral delivery system which facilitate cellular targeting. For example, a lipid encapsulated expression plasmid may incorporate modified surface cell receptor ligands to facilitate targeting. Although a simple liposome formulation may be administered, the liposomes either filled or decorated with a desired composition of the invention of the invention can delivered systemically, or can be directed to a tissue of interest, where the liposomes then deliver the selected therapeutic/immunogenic peptide compositions. Examples of such ligands includes antibodies, monoclonal antibodies, humanized antibodies, single chain antibodies, chimeric antibodies or functional fragments (Fv, Fab, Fab′) thereof. Alternatively, the DNA constructs of the invention can be linked through a polylysine moiety to a targeting moiety as described in Wu, et al. U.S. Pat. No. 5,166,320 issued Nov. 24, 1992 and Wu, et al, U.S. Pat. No. 5,635,383 issued Jun. 3, 1997.

In one embodiment of the invention as exemplified herein, the vector is a viral vector. The terms virus(es) and viral vector(s) are used interchangeably herein. The viruses useful in the practice of the present invention include recombinantly modified enveloped or non-enveloped DNA and RNA viruses, preferably selected from baculoviridiae, parvoviridiae, picornoviridiae, herpesveridiae, poxviridae, adenoviridiae, or picornnaviridiae. The viral genomes may be modified by conventional recombinant DNA techniques to provide expression of interferon and may be engineered to be replication deficient, conditionally replicating or replication competent. Chimeric viral vectors which exploit advantageous elements of each of the parent vector properties (See e.g., Feng, et al.(1997) Nature Biotechnology 15:866-870) may also be useful in the practice of the present invention. Minimal vector systems in which the viral backbone contains only the sequences needed for packaging of the viral vector and may optionally include an interferon expression cassette may also be employed in the practice of the present invention. In some instances it may be advantageous to use vectors derived from different species from that to be treated which possess favorable pathogenic features such as avoidance of pre-existing immune response. For example, equine herpes virus vectors for human gene therapy are described in W098/27216 published Aug. 5, 1998. The vectors are described as useful for the treatment of humans as the equine virus is not pathogenic to humans. Similarly, ovine adenoviral vectors may be used in human gene therapy as they are claimed to avoid the antibodies against the human adenoviral vectors. Such vectors are described in WO 97/06826 published Apr. 10, 1997.

Many viruses exhibit the ability to infect a broad range of cell types. However, in some applications it may be desirable to infect only a certain subpopulation of cells. Consequently, a variety of techniques have evolved to facilitate selective or “targeted” vectors to result in preferential infectivity of the mature viral particle of a particular cell type. Cell type specificity or cell type targeting may also be achieved in vectors derived from viruses having characteristically broad infectivities such as adenovirus by the modification of the viral envelope proteins. For example, cell targeting has been achieved with adenovirus vectors by selective modification of the viral genome knob and fiber coding sequences to achieve expression of modified knob and fiber domains having specific interaction with unique cell surface receptors. Examples of such modifications are described in Wickham, et al. (1997) J. Virol. 71(11):8221-8229 (incorporation of RGD peptides into adenoviral fiber proteins); Arnberg, et al. (1997) Virology 227:239-244 (modification of adenoviral fiber genes to achieve tropism to the eye and genital tract); Harris and Lemoine (1996) TIG 12(10):400-405; Stevenson, et al. (1997) J. Virol. 71(6):4782-4790; Michael, et al. (1995) gene therapy 2:660-668 (incorporation of gastrin releasing peptide fragment into adenovirus fiber protein); and Ohno, et al. (1997) Nature Biotechnology 15:763-767 (incorporation of Protein A-IgG binding domain into Sindbis virus). Other methods of cell specific targeting have been achieved by the conjugation of antibodies or antibody fragments to the envelope proteins (see, e.g. Michael, et al. (1993) J. Biol. Chem. 268:6866-6869, Watkins, et al. (1997) Gene Therapy 4:1004-1012; Douglas, et al. (1996) Nature Biotechnology 14: 1574-1578. Viral vectors encompassing one or more of such targeting modifications may optionally be employed in the practice of the present invention to enhance the selective infection and expression of interferon in lung tissues, especially epithelial tissues of the lung.

In one embodiment of the invention as exemplified herein, the vector is an adenoviral vector. The term adenoviral vector refers collectively to animal adenoviruses of the genus mastadenovirus including but no limited to human, bovine, ovine, equine, canine, porcine, murine and simian adenovirus subgenera. In particular, human adenoviruses includes the A-F sugenera as well as the individual serotypes thereof the individual serotypes and A-F subgenera including but not limited to human adenovirus types 1, 2, 3, 4, 4a, 5, 6, 7, 8, 9, 10, 11 (Ad11A and Ad 11P), 12, 13,14,15,16,17,18,19, 19a, 20, 21, 22, 23, 24,25, 26, 27, 28, 29, 30, 31, 32, 33, 34, 34a, 35, 35p, 36, 37, 38, 39, 40, 41, 42, 43, 44, 45, 46, 47, 48, and 91. The term bovine adenoviruses includes, but is not limited to, bovine adenovirus types 1,2,3,4,7, and 10. The term canine adenoviruses includes but is not limited to canine types 1 (strains CLL, Glaxo, RI261, Utrect, Toronto 26-61) and 2. The term equine adenoviruses includes, but is not limited to, equine types 1 and 2. The term porcine adenoviruses includes but is not limited to porcine types 3 and 4. The use of adenoviral vectors for the delivery of exogenous transgenes are well known in the art. See e.g., Zhang, W-W. (1999) Cancer Gene Therapy 6:113-138. In one embodiment the adenoviral vector for expression of the interferon sequence is a replication deficient human adenovirus of serotype 2 or 5 created by elimination of adenoviral E1 genes resulting in a virus which is substantially incapable of replicating in the target tissues, optionally including a deletion of the protein IX function. A preferred recombinant viral vector is the adenoviral vector delivery system which has a deletion of the protein IX gene. See such systems disclosed in U.S. Pat. No. 6,210,939 which is assigned to the same assignee as the present invention and is herein incorporated by reference in its entirety for all purposes.

Preferred vectors are derived from the adenoviral, adeno-associated viral and retroviral genomes. In the most preferred practice of the invention, the vectors are derived from the human adenovirus genome. Preferred vectors are derived from the human adenovirus serotypes 2 or 5. The replicative capacity of such vectors may be attenuated (to the point of being considered “replication deficient”) by modifications or deletions in the E1a and/or E1b coding regions. Other modifications to the viral genome to achieve particular expression characteristics or permit repeat administration or lower immune response are preferred.

Alternatively, the viral vectors may be conditionally replicating or replication competent. Conditionally replicating viral vectors are used to achieve selective expression in particular cell types while avoiding untoward broad spectrum infection. The viral genome may be modified to include inducible promoters which achieve replication or expression only under certain conditions. Examples of inducible promoters are known in the art (See, e.g. Yoshida and Hamada (1997) Biochem. Biophys. Res. Comm. 230:426-430; Iida, et al. (1996) J. Virol. 70(9):6054-6059; Hwang, et al.(1997) J. Virol 71(9):7128-7131; Lee, et al. (1997) Mol. Cell. Biol. 17(9):5097-5105; and Dreher, et al.(1997) J. Biol. Chem. 272(46); 29364-29371. The viruses may also be designed to be selectively replicating viruses such as those described in Ramachandra, et al. PCT International Publication No. WO 00/22137, International Application No. PCT/US99/21452 published Apr. 20, 2000 and Howe, J., PCT International Publication No. WO WO00022136, International Application No. PCT/US99/21451 published Apr. 20, 2000. The virus may also be modified to be attenuated for replication in certain cell types. For example the adenovirus dl1520 containing a specific deletion in the E1b55K gene (Barker and Berk (1987) Virology 156: 107) has been used with therapeutic effect in human beings. Such vectors are also described in McCormick (U.S. Pat. No. 5,677,178 issued Oct. 14, 1997) and McCormick, U.S. Pat. No. 5,846,945 issued Dec. 8, 1998.

The vectors of the present invention may be modified to encode an additional transgene to the interferon gene, either in tandem through the use of IRES elements or through independently regulated promoters.

The present invention provides a delivery enhancing compound that, when formulated with a protein (e.g., an interferon) or nucleic acid enhances the delivery of the protein or nucleic acid to the epithelium of the target tissue or organ. “A delivery-enhancing compound” or “delivery enhancing agent” includes any compound that enhances delivery of the protein or nucleic acid to the epithelium or urothelium upon intravesicular administration. With respect to proteins, enhanced delivery can be ascertained by either or both of an increase in the amount of the protein which contacts the cells of the epithelium of the target tissue or organ or which enters the cells of the the epithelium (e.g., urothelium). The determination of whether a particular compound is effective in enhancing delivery of a proteins can be readily ascertained by those of ordinary skill in the art. The retention of protein by the urothelium may be measured by any means, for instance, ELISA, detection of fluorescent labels, modulation of cellular activity including gene expression.

With respect to gene delivery systems, the present invention provides a delivery enhancing compound that enhances the delivery of the interferon gene to the target cell, tissue, or organ. Enhanced delivery can be ascertained by either or both of an increase in the number of copies of the interferon gene or gene delivery system that enter each cell or a increase in the proportion of cells in, for example, a tissue or organ, that take up the interferon gene or interferon gene delivery system. Administration of an interferon gene delivery system to a tissue or organ in conjunction with a delivery enhancing compound results in an increase in the amount of the interferon gene that is delivered and expressed within the cells, relative to the amount of the gene delivered and expressed in the cells when administered in the absence of the delivery enhancing compound. The determination of whether a particular compound is effective in enhancing delivery of a nuclei acid delivery system by means known to those of skill in the art. To assess translation and protein expression, a reporter gene such as beta-galactosidase or green fluorescent protein may be incorporated into the nucleic acid delivery system to produce a readily assayable signal to assess the level of enhanced gene expression. To assess transcription and replication, the presence of copies of DNA may be assessed by PCR analysis.

Certain delivery enhancing compounds may possess asymmetric carbon atoms (optical centers) or double bonds; the racemates, diastereomers, geometric isomers and individual isomers are all intended to be encompassed within the scope of the present invention. Single diastereomer of pairs of enantiomers, for example, can be obtained by fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent. Alternatively, any enantiomer of an inventive compound may be obtained by stereospecific synthesis using optically pure starting materials or reagents of known configuration.

Delivery enhancing compounds may exist with different points of attachment of hydrogen, referred to as tautomers. Such an example may be a ketone and its enol form known as keto-enol tautomers. The individual tautomers as well as mixture thereof are encompassed by the inventive formulas.

The delivery enhancing compounds may have unnatural ratios of atomic isotopes at one or more of their atoms. For example, the compounds may be radiolabeled with isotopes, such as tritium or carbon-14. All isotopic variations of the compounds of the present invention, whether radioactive or not, are within the scope of the present invention. Where it is described in a compound formula that a “group” or “moiety” is attached to another portion of the compound, it is to be understood that a radical corresponding to the “group” or “moiety” wherein a H atom has been removed to form the radical is meant. The delivery enhancing compounds may be isolated in the form of their pharmaceutically acceptable acid addition salts, such as the salts derived from using inorganic and organic acids. Such acids may include hydrochloric, nitric, sulfuric, phosphoric, formic, acetic, trifluoroacetic, propionic, maleic, succinic, malonic and the like. In addition, certain compounds containing an acidic function can be in the form of their inorganic salt in which the counterion can be selected from sodium, potassium, lithium, calcium, magnesium and the like, as well as from organic bases. The term “pharmaceutically acceptable salts” refers to salts prepared from pharmaceutically acceptable non-toxic bases or acids including inorganic bases or acids and organic bases or acids.

Exemplary delivery-enhancing compounds are taught in U.S. Pat. No. 6,165,779, and U.S. patent application Ser. No. 08/889,355, filed on Jul. 8, 1997 and U.S. patent application Ser. No. 09/650,359, filed on Aug. 28, 2000, which are each assigned to the same assignee as the present application and are incorporated by reference in their entireties. Such compounds include, but are not limited to, detergents, alcohols, glycols, surfactants, bile salts, heparin antagonists, cyclooxygenase inhibitors, hypertonic salt solutions, and acetates. Alcohols include, for example, the aliphatic alcohols such as ethanol, N-propanol, isopropanol, butyl alcohol, acetyl alcohol. Glycols include, for example, glycerin, propyleneglycol, polyethyleneglycol and other low molecular weight glycols such as glycerol and thioglycerol. Acetates such as acetic acid, gluconic acid, and sodium acetate are further examples of delivery enhancing compounds. Hypertonic salt solutions such as 1M NaCl are also examples of delivery enhancing compounds. Examples of surfactants include sodium dodecyl sulfate (SDS) and lysolecithin, polysorbate 80, nonylphenoxy-polyoxyethylene, lysophosphatidylcholine, polyethyleneglycol 400, polysorbate 80, polyoxyethylene ethers, polyglycol ether surfactants and DMSO. Bile salts such as taurocholate, sodium tauro-deoxycholate, deoxycholate, chenodesoxycholate, glycocholic acid, glycochenodeoxycholic acid and other astringents like silver nitrate can also be used, as can heparin-antagonists like quaternary amines such as protamine sulfate. Cyclooxygenase inhibitors such as, for example, sodium salicylate, salicylic acid, and non-steroidal anti-inflammatory drugs (NSAIDS) such as indomethacin, naproxen, and diclofenac are also suitable.

Detergents that can function as delivery enhancing compounds include, for example, anionic, cationic, zwitterionic, and nonionic detergents. Exemplary detergents include, but are not limited to, taurocholate, deoxycholate, taurodeoxycholate, cetylpyridium, benalkonium chloride, ZWITTERGENT®3-14 detergent, CHAPS (3-[(3-Cholamidopropyl)dimethylammoniol]-1-propanesulfonate, hydrate, Aldrich), Big CHAP, Deoxy Big CHAP, TRITON®-X-100 detergent, C12E8, Octyl-B-D-Glucopyranoside, PLURONIC®-F68 detergent, TWEEN® 20 detergent, and TWEEN® 80 detergent (CALBIOCHEM® Biochemicals).

One example of a preferred delivery enhancing compound is, for example, is Big CHAP, which is a cholate derivative (see, e.g., Helenius et al. (I 979) “Properties of Detergents,” In: Methods in Enzymology, Vol.66, 734-749. Especially preferred delivery enhancing compounds are compounds of Formulae I, II, III, and VI, and VII and their pharmaceutically acceptable salts.

In additional embodiments, the invention provides compositions comprising the interferon and at least one delivery enhancing compound that has a Formula I: 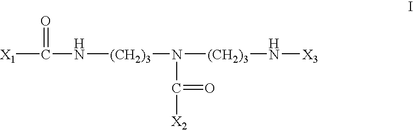
in which X₁ is and X₂ are selected from the group consisting of 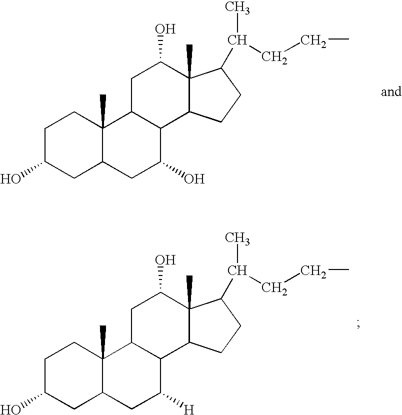
and X₃ is a saccharide group wherein the saccharide group can be selected from the group consisting of pentose monosaccharide groups, hexose monosaccharide groups, pentose-pentose disaccharide groups, hexose-hexose disaccharide groups, pentose-hexose disaccharide groups, and hexose-pentose disaccharide groups. Other saccharide groups can include more than one monosaccharide linked in either homo-oligosaccharides or hetero-oligosaccharides. Preferred monosaccharides include pentose and/or hexose residues. For example, the saccharide groups can be selected from the group consisting of pentose monosaccharide groups, hexose monosaccharide groups, pentose-pentose disaccharide groups, hexose-hexose disaccharide groups, pentose-hexose disaccharide groups, and hexose-pentose disaccharide groups. One example of a preferred saccharide group for X₃ is lactose.

In some embodiments, the delivery enhancing compounds of Formula I have X₃ saccharide groups that are composed of three or more monosaccharides. Preferably, the saccharide group has between one and eight monosaccharides, more preferably between one and four monosaccharides, and most preferably about two to three monosaccharides. The use of a trisaccharide, for example, can provide a compound having increased solubility.

In a further embodiment, X₁ and X₂ are both 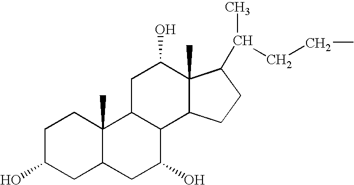
and X₃ is a glucose group.

Exemplary delivery enhancing compounds for use according to the invention include compounds of Formula II: 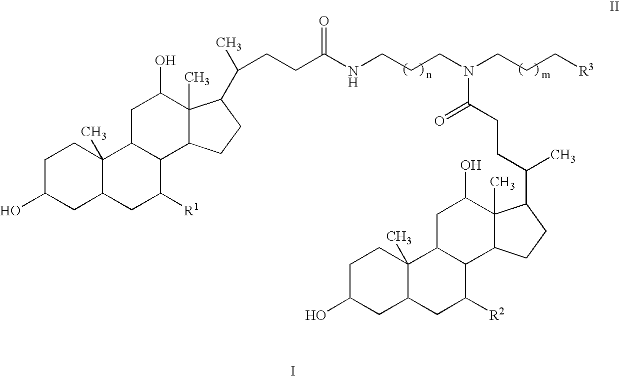
in which R¹ and R² are each independently a member selected from the group consisting of hydrogen, and a hydroxyl group; m and n are each independently selected from about 0-2; R³ is selected from the group consisting of —NR⁴R⁵ wherein R⁴ and R⁵ are each independently a member selected from the group consisting of a hydrogen, a saccharide residue, an optionally substituted alkyl, an optionally substituted acyl, and an optionally substituted acyloxy, and a quaternary ammonium salt —NR⁶R⁷R⁸X wherein R⁶, R⁷ and R⁸ are independently a member selected from the group consisting of hydrogen and C₁-C₄ alkyl, and X is the negatively charged ionically bound counterion selected from the group consisting of pharmacologically acceptable counterions. In some embodiments, the counter ion is a halogen or an optionally substituted carboxylate.

In one embodiment, the exemplary compound of Formula II has R¹ and R² which are both hydroxyl groups. In another embodiment, the compound of Formula II is one in which m and n each equal to 1. In another embodiment, the compound of Formula II is one in which R⁴ is hydrogen; and R⁵ is a member selected from the group consisting of a hydrogen, a saccharide residue, an optionally substituted alkyl, an optionally substituted acyl, and an optionally substituted acyloxy.

In one embodiment, the delivery enhancing compound has formula VI:

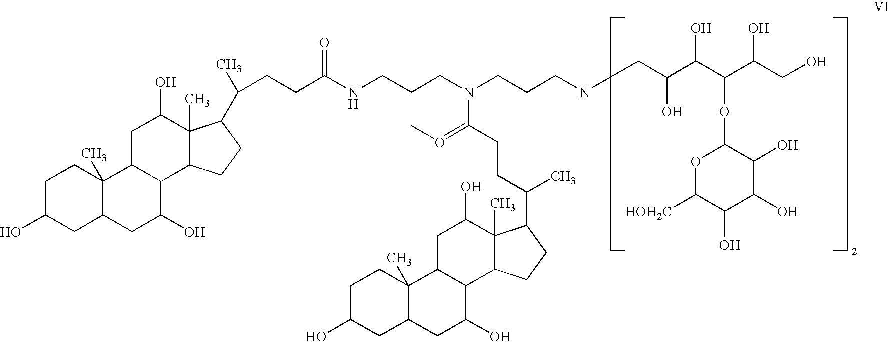

In another embodiment, the delivery enhancing compound has Formula VII (SYN3):

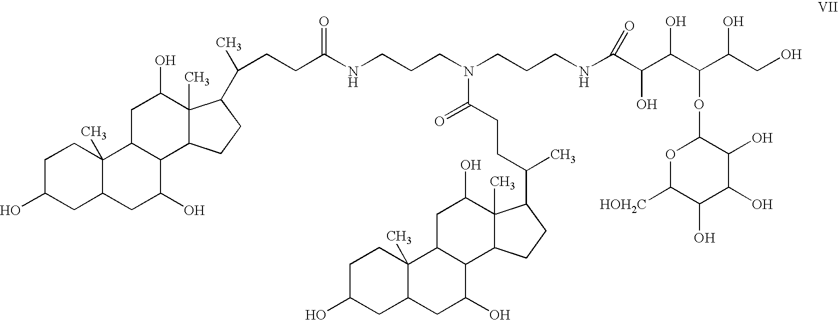

In another embodiment, at least one of R⁴ or R⁵ is succinyl or acetyl. In another embodiment, R³ is a trimethylammonium salt.

In another embodiment, R⁴ and R⁵ are each independently a member selected from the group consisting of a hydrogen, an optionally substituted alkyl, an optionally substituted acyl, and an optionally substituted acyloxy, and a quaternary ammonium salt —NR⁶R⁷R⁸X wherein R⁶, R⁷ and R⁸ are independently a member selected from the group consisting of hydrogen and C₁-C₄ alkyl, and X is the negatively charged ionically bound counterion selected from the group consisting of pharmacologically acceptable counterions.

One example of a preferred delivery enhancing compound for use in the methods and compositions is SYN3, which has Formula VII. Methods of making SYN3 and its homologues are taught in U.S. Pat. No. 6,392,069 which is assigned to the same assignee as the present application and incorporated by reference in its entirety.

For some applications, it is desirable to use delivery enhancing compounds in the methods and compositions of the invention that exhibit increased water solubility and/or delivery enhancing activity compared to other compounds. Exemplary delivery enhancing compounds are of Formula I in which R is a cationic group. Suitable cationic groups include, for example, tetramethyl and ammonium moieties, and salts thereof.

In another aspect, the invention provides a pharmaceutical composition for intravesicular administration of a protein to the bladder which comprises the protein and a delivery enhancing agent. In some embodiments, the delivery enhancing agent comprises SYN3 or a SYN3 homolog. In some embodiments, the delivery enhancing agent is SYN3 or a SYN3 homolog. In some embodiments, the delivery enhancing agent is SYN3 and the protein is an interferon. In some embodiments, the composition comprises an interferon and SYN3 (e.g., an interferon, about 0.1 to 10 mg/ml SYN3, a SYN3 solubilizing agent (e.g., a surfactant, including Big CHAP or hydroxy propyl beta cyclodextrin (HPβCG) or TRITON™-X-100 detergent/an octylphenoxypolyethoxy ethanol), a buffer and pharmacologically acceptable excipients. In some embodiments, the interferon is an alpha-interferon, beta-interferon, gamma-interferon, or omega-interferon or a fusion thereof. In one embodiment, the amount of SYN3 in the formulation is 1-10 mg/ml. The compositions may be lyophilized for dilution with a suitable pharmaceutical carrier prior to use.

In some embodiments, the invention also provides a pharmaceutical composition that contains the protein and a delivery enhancing compound. The concentration of the delivery enhancing compound in a formulation will depend on a number of factors such as the particular protein and the delivery enhancing compound being used, the buffer, pH, and administration protocol. The concentration of the delivery enhancing compound can be depend substantially upon the protein and its solubility. For agents effective at higher concentrations, the concentrations will often be in the range of 1% to 50% (v/v), preferably 10% to 40% (v/v) and most preferably 15% to 30% (v/v). For delivery enhancing agents effective at lower concentrations, the particular delivery enhancing compound of the invention may be preferably used in the range of about 0.002 to 2 mg/ml, more preferably about 0.02 to 2 mg/ml, most preferably about 0.1 to 1 mg/ml in the formulations of the invention. The delivery enhancing compounds of the invention which comprise SYN3 or its homologs or analogs are preferably used in the range of about 0.002 to 20 mg/ml, more preferably about 0.02 to 2 mg/ml, most preferably about 0.1 to 1 mg/ml in the formulations of the invention.

In another aspect, the invention provides a pharmaceutical formulation for administration of a recombinant adenovirus encoding an interferon gene and a delivery enhancing agent. In some embodiments, the delivery enhancing agent comprises SYN3 or a SYN3 homolog. In some embodiments, the gene delivery system comprises about 10⁹-10¹² particles (PN)/ml recombinant adenovirus encoding an interferon gene, about 0.1 to 10 mg/ml SYN3, a SYN3 solubilizing agent (e.g., a surfactant, including Big CHAP or hydroxy propyl beta cyclodextrin (HPβCG) or TRITON™-X-100 detergent/ an octylphenoxypolyethoxy ethanol), a buffer and pharmacologically acceptable excipients. In some embodiments, the interferon gene is an alpha-interferon, beta-interferon, gamma-interferon, or omega-interferon gene or a fusion thereof or a portion thereof encoding a polypeptide with an interferon anti-cancer, anti-infective, or immune system modulating bioactivity. In one embodiment, the amount of SYN3 in the formulation is 1-10 mg/ml. The compositions may be lyophilized for dilution with a suitable pharmaceutical carrier prior to use.

In some embodiments, the invention also provides a formulation that contains the interferon gene delivery system and a delivery enhancing compound. The concentration of the delivery enhancing compound in a formulation will depend on a number of factors such as the particular gene deliver system and delivery enhancing compound being used, the buffer, pH, target tissue or organ and mode of administration. The concentration of the delivery enhancing compound will depend substantially upon the agent used. For agents effective at higher concentrations, the concentrations will often be in the range of 1% to 50% (v/v), preferably 10% to 40% (v/v) and most preferably 15% to 30% (v/v). For delivery enhancing agents effective at lower concentrations, the particular delivery enhancing compound of the invention may be preferably used in the range of about 0.002 to 2 mg/ml, more preferably about 0.02 to 2 mg/ml, most preferably about 0.1 to 1 mg/ml in the formulations of the invention. The delivery enhancing compounds of the invention which comprise SYN3 or its homologs or analogs are preferably used in the range of about 0.002 to 20 mg/ml, more preferably about 0.02 to 2 mg/ml, most preferably about 0.1 to 1 mg/ml in the formulations of the invention.

The delivery enhancing compounds for use in the methods and compositions of the invention are typically formulated in a solvent in which the compounds are soluble, although formulations in which the compounds are only partially solubilized are also suitable. Phosphate buffered saline (PBS) is one example of a suitable solubilizing agent for these compounds, and others are known to those of skill in the art. One will recognize that certain additional excipients and additives may be desirable to achieve solubility characteristics of these agents for various pharmaceutical formulations. For example, well known solubilizing agents such as detergents, fatty acid esters, and surfactants can be added in appropriate concentrations so as to facilitate the solubilization of the compounds in the various solvents to be employed. Where the formulation includes a detergent, the detergent concentration in the final formulation administered to a patient is preferably about 0.5-2× the critical micellization concentration (CMC). Suitable detergents include those listed above. The identification of suitable detergents and appropriate concentrations for their use can be determined as described herein. One example of a solubilizing agent for compounds such as SYN3 and related compounds is Tween 80 at a concentration of approximately 0.05% to about 0.3%, more preferably at a concentration of about 0.10% to about 0.15%. Big CHAP is also a solubilizing agent for SYN3 and related compounds.

When used for pharmaceutical purposes, the formulations of the invention include a buffer that contains the delivery-enhancing compound. The buffer can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al., Biochemistry, (1966) 5:467. The pH of the buffer in the pharmaceutical composition comprising a modulatory therapeutic protein for example, is typically in the range of 6.4 to 8.4, preferably 7 to 7.5, and most preferably 7.2 to 7.4.

The compositions of this invention can additionally include a protein stabilizer or solubilizer, enhancer or other pharmaceutically acceptable carriers or vehicles. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. One skilled in the art would know that the choice of pharmaceutically acceptable carrier depends on the route of administration and the particular physio-chemical characteristics of the therapeutic protein. Examples of carriers, stabilizers or adjuvants can be found in Martin, Remington's Pharm. Sci., 15th Ed. (Mack Publ. Co., Easton, Pa. 1975), and Remington: The Science and Practice of Pharmacy (19th Ed) (Lippincott, Williams & Wilkins Publisher, December 1995) which are incorporated herein by reference.

A further aspect of the invention is a pharmaceutical composition comprising an interferon and SYN3 in combination with a pharmaceutically acceptable aqueous carrier, at least one pharmaceutically acceptable solubilizer, and at least one pharmaceutically acceptable bulking agent. Such pharmaceutical compositions may further comprise SYN3 in combination with a pharmaceutically acceptable carrier and the protein. See U.S. patent application Ser. No. 10/329,043 [titled “SYN3 COMPOSITIONS AND METHODS” Attorney Docket No. 016930-000841US] filed on Dec. 20, 2002 and assigned to the same assignee as the present application and hereby incorporated by reference in its entirety.

An exemplary formulation for administration of a recombinant adenovirus is about 10⁹-10¹¹ PN/ml virus, about 0.1 to 1 mg/ml SYN3 with a suitable SYN3 solubilizing agent (e.g., about 2-10 mM Big CHAP or about 0.1-1.0 mM TRITON®-X-100) detergent, in phosphate buffered saline (PBS), plus about 2-3% sucrose (w/v) and about 1-3 mM MgCl.sub.2, at about pH 6.4-8.4.

A further aspect of the invention is a pharmaceutical composition comprising an interferon gene delivery system and SYN3 in combination with a pharmaceutically acceptable aqueous carrier, at least one pharmaceutically acceptable solubilizer, and at least one pharmaceutically acceptable bulking agent. Such pharmaceutical compositions may further comprise SYN3 in combination with a pharmaceutically acceptable carrier and an expression vector or gene delivery system comprising an interferon DNA sequence inserted into the vector to which the interferon DNA is foreign. The nucleic acid sequence of the interferon may, in some embodiments, may be foreign to or not otherwise significantly expressed in the intended target tissue or organ of the subject. See U.S. patent application Ser. No. 10/329,043 [titled “SYN3 COMPOSITIONS AND METHODS” Attorney Docket No. 016930-000841US] filed on Dec. 20, 2002 and assigned to the same assignee as the present application and hereby incorporated by reference in its entirety.

Solvents that may be used for the formulations of the present invention include, for example, aqueous solvents such as water for injection, and/or nonaqueous solvents, such as DMSO and N,N-Dimethyylacetamide, also known as DMA, and co-solvent mixtures, e.g., glycerol and water, as prepared preferably in accordance with USP standards.

The formulations preferably contain polysorbates, or polyoxyethylene sorbitan esters, a class of nonionic surfactants that included, e.g., polysorbate 80 and polysorbate 20, amongst others, Pluronics, or polyethylenepolypropylene glycol block copolymers, a class of nonionic surfactants, that include, e.g., Pluronic L68 and L92, amongst others, and hydroxypropyl-beta-cyclodextrin, a polysubstituted hydroxyalkyl-beta-cyclodextrin, which is a class of nonionic complexing agents, that include, e.g., HPβCD and Big CHAP. Preferred are HPβCD, Big CHAP, Polysorbate 80, Polysorbate 20, Pluronic L64, and Pluronic L92 as solubilizing agents. The solubilizers can be used, for example, either individually or in combinations. The concentrations of the solubilizing agents are set forth below. HPβCD can be present in a concentration of about 50 to 500 mg/ml, Big CHAP can be present in a concentration of about 20 to about 360 mg/ml, Polysorbate 80 can be present in a concentration of about 1 to 36 mg/ml, the Pluronics can be present in concentrations of about 1 to about 150 mg/ml, and the other ingredients may be present in concentrations as set forth below.

The lyophilized formulations of SYN3 preferably contain a citrate buffering system. More preferably, the citrate buffering system can comprise at least one citric buffer, such as citric acid monohydrate USP or sodium citrate dihydrate USP. More preferably, the citrate buffering system comprises a combination of citric acid monohydrate USP and sodium citrate dihydrate USP. When used in combination, the amount of citric acid monohydrate USP can be present in a concentration of about 0.005 to about 2 mg/ml, more preferably 0.016 to about 1.35 mg/ml, preferably 0.016 to about 0.72 mg/ml, preferably about 0.005 to about 1.35, and the sodium citrate dihydrate USP can be present in a concentration of about 0.02 to about 5.37 mg/ml, preferably 0.05 to 3.00 mg/ml, preferably 0.05 to 2.31 mg/ml. Other suitable buffering systems that are suitable include, for example, phosphate, glycine, either in place of the citrate buffering system or in combination therewith, and varying combinations of all of the above.

The buffering system will provide a pH of the lyophilized formulation such that there is improved stability. Preferably, the pH will be in a range of about 5 to about 6. The admixture aqueous formulations of SYN3 are preferably buffered at about a pH of about 7 to about 8.5, preferably about 7.4, and SYN3 remains stable in the dehydrated powder for at least 3 months at 40° C.

The lyophilized formulations preferably contain glycine or mannitol as freeze-drying bulking agents. Other suitable freeze-drying bulking agents that may be used include, for example, lactose, recombinant gelatin, and methylcellulose. The freeze drying-bulking agent may be present in a concentration of from about 5 to 100 mg/ml when the agent is mannitol, and about 10 to 200 mg/ml when the agent is glycine.

The lyophilized formulations preferably contain ascorbic acid as an antioxidant. Other suitable antioxidants that may be used include, for example, citric acid. When ascorbic acids is the antioxidant, it may be present in a concentration of about 0.001 to about 0.6 mg/ml.

The compositions of this invention may additionally include, for example, a stabilizer, enhancer or other pharmaceutically acceptable carriers or vehicles. A pharmaceutically acceptable carrier can contain a physiologically acceptable compound that acts, for example, to stabilize the recombinant adenoviral vector delivery system comprising the tumor suppressor gene. A physiologically acceptable compound can include, for example, carbohydrates, such as glucose, sucrose or dextrans, Hydroxypropyl-β-Cyclodextrin, antioxidants, such as ascorbic acid or glutathione, chelating agents, low molecular weight proteins or other stabilizers or excipients.

Other physiologically acceptable compounds include, for example, wetting agents, emulsifying agents, dispersing agents or preservatives, which are particularly useful for preventing the growth or action of microorganisms. Various preservatives are well known and include, for example, phenol and ascorbic acid. One skilled in the art would know that the choice of pharmaceutically acceptable carrier, depends on the route of administration and the particular physiochemical characteristics of the therapeutic protein. Examples of carriers, stabilizers or adjuvants can be found in Gennaro, Remington's: The Science and Practice of Pharmacy, 19th Ed. (Mack Publishing. Co., Easton, Pa. 1995), incorporated herein by reference.

The subjects of the invention are mammals, including, but not limited to, mice, rats, primates, and particularly, humans.

Exemplary delivery enhancing compounds for both nucleic acid and protein delivery include homologs of SYN3 for use according to the invention include compounds of Formula II.

In some embodiments, the compositions of the invention comprise a “therapeutically effective” amount of a therapeutic agent (e.g., the protein or gene delivery system for the protein) in a buffer comprising a delivery-enhancing compound. “Therapeutically effective” as used herein refers to the prevention of, reduction of, or curing of a disease state such as cancer or viral infection or the symptoms thereof.

The delivery enhancing compounds may be applied singly or in combination with an interferon or an interferon gene delivery system or in further combination with additional agents. For instance, the formulations are useful in treating diseases and conditions, including cancers, cellular proliferative disorders, and chronic infections. “Cancer” generally pertains to a unrestrained proliferation of cells within a tissue or organ. It is intended to encompass conditions in which one or more cells is classified as cancerous, malignant, tumorous, precancerous, transformed, or as an adenoma or carcinoma, or any other synonym commonly used in the art for these conditions.

Therapeutically effective amounts of the pharmaceutical composition comprising the interferon gene in a recombinant viral vector delivery system formulated in a buffer comprising a delivery-enhancing agent can be administered in accord with the teaching of this invention. For example, therapeutically effective amounts of the interferon gene in the recombinant adenoviral vector delivery system formulated in a buffer containing a delivery-enhancing agent are in the range of about 10⁸ particles/ml to 10¹² particles/ml, more typically about 10⁸ particles/ml to 5×10¹¹ particles/ml, most typically 10⁹ particles/ml to 10¹¹ particles/ml (PN/ml) or 10¹¹ particles/ml to 10¹¹ particles/ml (PN/ml). The foregoing dosages are generally administered as frequently as tolerated by the subject. It is common practice in the field of oncology to provide dosages at or near the maximum tolerated dose.

Therapeutically effective amounts of the pharmaceutical composition comprising the protein (e.g., interferon) formulated in a buffer comprising a delivery-enhancing agent can be administered in accord with the teaching of this invention. For example, therapeutically effective amounts of the interferon or interferon gene delivery system as may be readily determined by one of ordinary skill in the art. The dosages are generally administered as frequently as tolerated by the subject. As noted above, it is common practice in the field of oncology to provide dosages at or near the maximum tolerated dose.

In one embodiment of the invention for the treatment of bladder cancer, the interferon is provided in conjunction with a delivery enhancing agent as part of a multi-course treatment regimen consisting of three weekly cycles of treatment, each cycle providing a single intravesical administration on day 1 or optionally on days 1 and 2 of each cycle. The typical manner of treatment of bladder cancer, a practitioner would insert a catheter in the urethra allowing the subject to void. A solution is prepared comprising the formulated delivery enhancing agent and the therapeutic interferon protein or interferon gene delivery system and instilled into the bladder. The catheter is clamped so as to maintain the solution in the bladder for approximately one hour. It should be noted that longer exposure may be desirable to enhance the delivery to the epithelium but practical considerations generally limit such practice. For example, one hour is generally the point at which the subject will feel the need to void. The effect of the delivery enhancing agent has been demonstrated to produce a prolonged effect such that the pre-exposure of the bladder to the delivery enhancing agent in advance of the instillation of the protein will provide an enhanced delivery. This may be employed where compatibility between the protein formulation and the delivery enhancing agent formulation is a concern. Following the desired exposure, the catheter clamp is removed from the catheter and the subject allowed to void. The foregoing procedure may be repeated as many times as tolerated by the patient to achieve a therapeutic effect. The therapeutic effect may be readily assessed by conventional means such as a cystoscope inserted into the bladder.

These formulations can be administered in a volume corresponding to the size of the void to be filled or organ or tissue to be treated. For the human bladder, a suitable volume of administration will depend upon the age and size of the subject, and particularly of their bladder. Administered volumes may typically range from 50 to 500 ml, and more commonly from 50 to 250 ml. or from 100 to 200 ml. In some methods of administration, the amount to be administered can be economically conserved by inserting a balloon catheter into the vesical or bladder to be treated and inflating the balloon portion of the catheter so as to reduce the void volume to be occupied by the administered composition.

In addition, the above treatment regimens may be supplemented or used in conjunction with other therapeutic treatment regimens to provide an enhanced therapeutic effect. The methods of the present invention may be supplemented for example by the conventional BCG and/or chemotherapeutic treatment regimens. It is standard of practice in the field of oncology to provide multiple treatment regimens to an individual to maximize the effect.

In another aspect, the invention provides a kit having the delivery enhancing compound in a first container and the protein or gene delivery system in a second container. In one embodiment, the contents of the containers are combined in preparing a formulation comprising both the delivery enhancing agent and the protein or gene delivery system which is thereafter to be administered to a subject. In another embodiment, the contents of the containers are administered as separate preparations to a patient. The delivery enhancing agent may be administered before, during or after administration of the protein or gene delivery system. In an exemplary embodiment, they are administered concurrently or nearly concurrently. The formulations of either one or both of the containers may be a lyophilized powder to be brought up in a pharmaceutically acceptable liquid carrier.

In another embodiment, the kit provides a first container containing SYN3 or a SYN3 homologue or analog capable of enhancing the delivery of a protein or a gene delivery system; and a second container containing the protein or the gene delivery system. In one further embodiment, the first container contains a lyophilized formulation of SYN3 or a SYN3 homologue or analog. In another embodiment, the second container contains a lyophilized formulation of the protein. In a further embodiment, both the first and second container hold a lyophilized formulation of their respective contents. In an exemplary further embodiment, the protein is, or the gene delivery system encodes a protein that is, a polypeptide which is substantially identical to a human alpha-interferon polypeptide or is a human alpha-interferon polypeptide.

In some embodiments, the administrations according to the methods and compositions of the invention may be repeated over various intervals of time so as to increase the therapeutic effect. These intervals may be approximately daily, weekly, or monthly. The number of administrations will vary with the therapeutic regime and its duration. Generally, therapeutic administrations according to the invention may be given semi-weekly, weekly, biweekly, monthly, or bimonthly for up to 2 months, four months, six months or even longer. The frequency of administration may be tailored to the individual subject by monitoring the production or release of interferon in the subject or assessing the clinical responses of the patient (e.g., tumor reduction if the disease being treated is cancer).

In another embodiment where the condition to be treated is cancer ofthe urinary bladder and the therapeutic protein (e.g., an interferon) or gene delivery system (e.g. for a gene encoding an interferon) is administered intravesically, the response of a subject to the therapeutic methods and compositions of the present invention is assessed by monitoring the size and number of tumor masses found in the urinary bladder. This monitoring may by measurement of tumor specific antigens or products in bodily fluids such as blood or urine or by direct imaging methods such as MRI or radioisotope imaging techniques.

Compositions formulated with the delivery-enhancing agents and an interferon or a gene encoding an interferon can be used to provide interferon to cells, including in particular, the epithelial cells of organs and tissues. The therapeutic effects and uses of interferon are known to one of ordinary skill in the art. For instance, compositions according to the invention are useful in the treatment of cancer. Cancerous tissues and organs suitable for treatment according to the inventive methods and compositions include any tissue or organ having an epithelial membrane such as the gastrointestinal tract, the bladder, respiratory tract, and the lung. Examples include but are not limited to carcinoma of the bladder and upper respiratory tract, vulva, cervix, vagina, uterus or bronchi; local metastatic tumors of the peritoneum; broncho-alveolar carcinoma; pleural metastatic carcinoma; carcinoma of the mouth and tonsils; carcinoma of the nasopharynx, nose, larynx, esophagus, stomach, colon and rectum, gallbladder, or skin; or melanoma.

The methods and compositions of the invention, in particular, will be useful in the treatment of bladder cancer in mammals and, more particularly humans. In one embodiment, the methods and compositions of the invention are used to treat individuals who have been treated with a different bladder cancer therapy and failed to respond satisfactorily as measured by either a failure to reduce the size of or number of the bladder tumor masses or by a later relapse or resurgence or recurrence of such tumors. In one embodiment, the methods and compositions of the present invention are administered to subjects who have failed to respond satisfactorily to BCG therapy. In some embodiments, the methods and compositions of the present invention are combined with another cancer therapy such as BCG therapy in the treatment of bladder cancer. In such embodiments, subjects may receive the methods and compositions of the present invention before, after, or during their BCG therapy.

In some embodiments, the recurrence of bladder cancer or the efficacy of therapy may be assessed by cytoscopy or, more preferably, the measurement of microsatellite DNA in urine. See Steiner G, Schoenberg M P, Linn J F, Mao L, Sidransky D, “Detection of bladder cancer recurrence by microsatellite analysis of urine,” Nat Med., 1997 Jun;3(6):621-4. In one embodiment for the treatment of bladder cancer, the therapeutic composition is administered via a balloon catheter. The catheter has a balloon portion which can be inserted via the urethra to the bladder void space where it is inflated to occupy a portion of the void space, preferably a majority of the space so as to leave a minimal void space to be occupied by the administered composition according to the invention. The catheter has another portion which transports the composition into the urinary bladder so as to come into contact with the bladder void space. Balloon urinary catheters are known to one of ordinary skill in the art.

In some embodiments, the compositions according to the invention can be administered in a way which reduces or avoids dilution of the delivery enhancing agent, or alternatively, the amount of the delivery enhancing agent in a preparation can be increased to take into account an expected dilution volume. Ways for avoiding dilution including emptying the target space by physiological means (e.g., urination, fasting) or pharmacological interventions (e.g., purgatives) or medical interventions (draining via catheters). Balloon catheters or other means may be used to isolate a volume of space within a lumen or passage to be occupied by the composition. In some embodiments, a balloon catheter can be used to also partially occupy the space to be filled so as to reduce the space needed to be occupied. In other embodiments, the formulation itself is formulated to act as a reservoir of the agent or to adhere to the surfaces of the target space and so avoid immediate dilution by bulk mixing or loss by bulk flow.

The synergy between the delivery enhancing agent and the interferon or interferon gene therapy upon their intravesical administration to the urinary bladder helps to reduce or avoid the side effects usually associated with interferon therapy.

Administration of an appropriate amount of the compositions may be by any means known in the art such as, for example, topical, local, oral or rectal, parenteral, intraperitoneal, intravenous, subcutaneous, subdermal, dermal, intranasal, ocular, pulmonary, transdermal or transurethral, intravesical, intramuscular, or transurethral and intravesical administration. In some embodiments, administration is transdermal. An appropriate amount or dose of the composition can be determined empirically as is known in the art.

The pharmaceutically or physiologically acceptable salts include, but are not limited to, metal salts such as sodium salt, potassium salt, lithium salt and the like; alkaline earth metals such as calcium salt, magnesium salt and the like; organic amine salts such as triethylamine salt, ethanolamine salt, triethanolamine salt, dicyclohexylamine salt, and the like; inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like.

### EXAMPLES

The following examples are provided for illustrative purposes, and are not intended to limit the scope of the invention as claimed herein. Any variations in the exemplified articles and/or methods which occur to the skilled artisan are intended to fall within the scope of the present invention.

### Example 1

U.S. Pat. No. 6,312,681 discloses a method for delivering an adenoviral vector which comprises a transgene to a cancer cell in the epithelial membrane of a bladder, the method comprising administering to the epithelial membrane the adenoviral vector and between 1% and 50% (v/v) ethanol or another delivery enhancing agent, wherein the adenoviral vector infects the cell and the transgene is expressed in infected cells. U.S. Pat. No. 6,312,681 assigned to the same assignee as the present application is hereby incorporated by reference in its entirety.

### Example 2

The following table represents exemplary ranges of the ingredients for non-aqueous liquid SYN3 formulations of the present invention. Prior to administration (e.g., for bladder cancer), the SYN3 solution is combined with the recombinant adenovirus preparation in a 1:50 v/v ratio to form an admixture that is administered to the patient.

| | Range of Ingredient Concentrations | | | |
|---|---|---|---|---|
| Ingredient | mg/ml | mg/ml | mg/ml | mg/ml |
| SYN3 | 0.001-150 | 0.001-150 | 0.001-150 | 0.001-150 |
| Polysorbate 20 | 0.001-150 | | | |
| Polysorbate 80 | | 0.001-150 | | |
| Pluronic L64 | | | 0.001-100 | |
| Pluronic L92 | | | | 0.001-100 |
| N,N-Dimethylacetamide | 1 ml | 1 ml | 1 ml | 1 ml |
| qs ad | | | | |

To prepare, weigh approximately 75% of DMA into a glass beaker. To a separate beaker, charge the surfactant (Polysorbate 80, Polysorbate 20, Pluronic L64 or Pluronic L92) and dissolve in a small volume (approximately 10% of final volume) of DMA. Charge the DMA/surfactant solution into the DMA with constant stirring. Preweigh SYN3 in a separate container. Slowly charge the SYN3 into the solution while stirring. Once the SYN3 is dissolved, add sufficient DMA to final volume by weight (density=0.962 g/ml at 25° C.). Filter the solution through a 0.22 filter attached to a syringe equipped with a nonlatex plunger and store the solution in a tightly sealed glass container at 4° C.

The following additional compositions may be prepared in accordance with Example 1.

| Ingredient | mg/ml | |
|---|---|---|
| SYN3 | 51 | |
| Polysorbate 20 | 50 | |
| N,N-Dimethylacetamide qs ad | 1 | ml |
| SYN3 | 51 | |
| Polysorbate 80 | 50 | |
| N,N-Dimethylacetamide qs ad | 1 | ml |
| SYN3 | 51 | |
| Pluronic L64 | 25 | |
| N,N-Dimethylacetamide qs ad | 1 | ml |
| SYN3 | 51 | |
| Pluronic L92 | 25 | |
| N,N-Dimethylacetamide qs ad | 1 | ml |

The following 25 ml batches were prepared according to the same procedure.

| Ingredient | mg/ml | |
|---|---|---|
| SYN3 | 1.275 | |
| Polysorbate 20 | 1.250 | |
| N,N-Dimethylacetamide qs ad | 25 | ml |
| SYN3 | 1.275 | |
| Polysorbate 80 | 1.250 | |
| N,N-Dimethylacetamide qs ad | 25 | ml |
| SYN3 | 1.275 | |
| Pluronic L64 | 0.625 | |
| N,N-Dimethylacetamide qs ad | 25 | ml |
| SYN3 | 1.275 | |
| Pluronic L92 | 0.625 | |
| N,N-Dimethylacetamide qs ad | 25 | ml |

### Example 3

The following table represents exemplary ranges of the ingredients for lyophilized SYN3 formulations of the present invention. The compounded solution is filled as indicated into a 20-ml capacity Type II glass vial and lyophilized. Preparation for administration requires addition of 20 ml of WFI to the vial containing the freeze-dried cake to redissolve the SYN3. The SYN3 solution is combined with p53, or any recombinant adenovirus preparation, in a v/v ratio of 1:5. The admixture is then administered to the patient for, for instance, bladder cancer.

| | Range of Concentrations | | |
|---|---|---|---|
| INGREDIENT | mg/ml | mg/ml | mg/ml |
| SYN3 | 0.001-150 | 0.001-150 | 0.001-150 |
| Citric Acid Monohydrate | 0.016-0.72 | 0.016-0.96 | 0.005-1.35 |
| Sodium Citrate Dihydrate | 0.05-2.31 | 0.05-3 | 0.02-5.37 |
| Hydroxypropyl-β-cyclodextrin | — | 50-500 | — |
| Big CHAP | 20-360 | — | — |
| Glycine | 10-200 | — | — |
| Mannitol | — | — | 5-100 |
| Polysorbate 80 | — | 1.0-36.0 | 10-200 |
| Ascorbic Acid^{a} | 0.001-0.6 | 0.001-0.6 | — |
| Water for Injection (WFI) | 1 ml | 1 ml | 1 ml |
| qs ad | | | |
| PH Range | 5-6 | 5-6 | 5-6 |
| ml Fill into 20-ml vial^{b} | 5.3 | 5.3 | 5.3 |
| Reconstitution Volume of | 20 ml | 20 ml | 20 ml |
| WFI^{c} | | | |

The following examples are methods of preparing the lyophilized formulations.

### Example 4

| Ingredient | Grams/Liter | |
|---|---|---|
| SYN3 | 24 | |
| Citric Acid Monohydrate USP | 0.24 | |
| Sodium Citrate Dihydrate USP | 0.77 | |
| Big CHAP | 120 | |
| Glycine USP | 50 | |
| Ascorbic Acid USP | 0.25 | |
| Water for Injection USP qs ad | 1000 | ml |

The actual amount of SYN3 to be charged will be adjusted according to the purity of the drug substance batch using the following formula:
grams SYN3=24.0×100/(% Purity).
For example:
SYN3 drug substance=97.0% pure.
24.0×100/97.0=24.7 grams SYN3 to be charged for a 1-Liter batch.

Accordingly, to determine the amount of SYN3 that will be charged to the batch according to the following formula:
g SYN3/Liter=24.0 g/Liter×[100/(% SYN3 Batch Purity)]

The volume of Water for Injection to be charged to the batch is to be determined according to the following formula:
Volume of Water for Injection (Liters)=Batch Volume (Liters)×0.5

Charge the volume of water for injection calculated using the formula above into a tared compounding vessel equipped with an agitator. Charge and dissolve, with agitation, the Big CHAP. Sterile Water for Injection may be used to rinse the weighing vessel to retrieve all of the material. Complete dissolution of Big CHAP may require approximately 30 to 60 minutes of continuous agitation at a moderate stirring rate. Charge and dissolve with agitation (moderate stirring rate) the SYN3 into the Big CHAP solution. Water for Injection may be used to rinse the weighing vessel to retrieve all of the material. Complete dissolution of SYN3 may require up to 1 hour of mixing. Charge and dissolve with agitation and in order: Glycine, Ascorbic Acid, Citric Acid Monohydrate and Sodium Citrate Dihydrate into the solution that contains both Big CHAP and SYN3. Water for Injection may be used to rinse the weighing vessels to retrieve all of the material. Add Water for Injection to bring the batch to the final volume (density of the solution is approximately 1.051 g/ml at 25° C.). Mix the solution for a minimum of 15 minutes.

Remove a small (<5 ml) sample of the solution for pH measurement. The pH should be between 5.0 and 6.0. No pH adjustment is necessary. One of ordinary skill in the art can readily ascertain the pH of the resulting product.

To complete compounding, aseptically filter the solution. If necessary, the compounded batch may be stored at 2° C. to 8° C. for up to 24 hours in a sealed, sterilized, stainless steel pressure vessel prior to filling into the vials. The batch may be filtered more than once to assure sterility.

Aseptically fill 5.3±0.1 ml of solution into 20-ml Type 1 flint glass vials that have been washed and sterilized. Aseptically insert 20-mm West 4416/50 1yo-shape rubber stoppers that have been washed, siliconized and sterilized into the vials in the lyophilization position.

Precool the lyophilizer shelves to 4±2° C. Aseptically load the trays of filled vials onto the lyophilizer shelves. After all the trays are loaded, cool the shelves to −40° C. in 1 hour and maintain the product at −35° C. or below for at least 4 hours before proceeding. Start cooling the condenser. When the condenser temperature is at −45° C. or below, begin evacuating the chamber. When 50-70 mm Hg of vacuum pressure is attained, heat the shelf temperature to −20° C. over 0.5 hour. Maintain the shelf temperature at −20° C. for 36 hours at approximately 150 mm Hg pressure (100 to 200 mm Hg pressure). Product temperature must remain at or above −20° C. for at least 6 hours before proceeding. Heat the shelf to 25° C. in 1 hour and reduce pressure to approximately 50 mm Hg pressure. Maintain the shelf temperature at 25° C. at approximately 50 mm Hg pressure for 14 hours. Vent the chamber with sterile filtered nitrogen to approximately 950 mm Hg. Stopper the vials inside the lyophilizer. Remove the vials from the lyophilizer and crimp the vials with 20-mm aluminum seals. The vials should be stored at 2° C. to 8° C. until inspection is completed.

The product is a white to off-white cake. The vials should be stored between 2° C. to 8° C. after inspection. For labeling and inspection purposes, the vials may be exposed to 19° C.-25° C. for up to 6 hours.

### Example 5

The following examples were prepared in accordance with the batch preparation as set forth in Example 4 above.

| Ingredient | mg/ml | |
|---|---|---|
| SYN3 | 24 | |
| Citric Acid Monohydrate USP | 0.24 | |
| Sodium Citrate Dihydrate USP | 0.77 | |
| Big CHAP | 120 | |
| Glycine USP | 50 | |
| Ascorbic Acid USP | 0.25 | |
| Water for Injection USP qs ad | 1 | Ml |

The pH of the resulting product was 5.34.

| Ingredient | mg/ml | |
|---|---|---|
| SYN3 | 24 | |
| Citric Acid Monohydrate USP | 0.32 | |
| Sodium Citrate Dihydrate USP | 1.02 | |
| HPβCD | 200 | |
| Polysorbate 80 | 12 | |
| Ascorbic Acid USP | 0.25 | |
| Water for Injection USP qs ad | 1 | ml |

The pH of the resulting product was 5.45.

| Ingredient | mg/ml | |
|---|---|---|
| SYN3 | 24 | |
| Citric Acid Monohydrate USP | 0.45 | |
| Sodium Citrate Dihydrate USP | 1.79 | |
| Mannitol | 30 | |
| Polysorbate 80 | 72 | |
| Water for Injection USP qs ad | 1000 | ml |

The pH of the resulting product was 5.76.

Stability testing of the resultant lyophilized products showed them to be highly stable with respect to SYN3. Stability testing was accomplished by HPLC.

Lyophilized formulations were reconstituted (redissolved) with 19.5 ml WFI: Samples were placed in the indicated temperature conditions, incubated for specified times and concentrations were determined by HPLC and compared to initial concentrations.

### Example 6

| Ingredient | mg/ml | |
|---|---|---|
| SYN3 | 24.0 | |
| Citric Acid Monohydrate USP | 0.32 | |
| Sodium Citrate Dihydrate USP | 1.02 | |
| Hydroxypropyl-β-cyclodextrin | 200 | |
| Polysorbate 80 | 12.0 | |
| Ascorbic Acid USP | 0.25 | |
| Water for Injection USP qs ad | 1000 | ml |

The actual amount of SYN3 to be charged will be adjusted according to the purity of the drug substance batch using the following formula:
grams SYN3=24.0×100/(% Purity).

Sample Calculation:
SYN3 drug substance=97.0% pure.
24.0×100/97.0=24.7 grams SYN3 to be charged for a 1-Liter batch.

The following Example was prepared as such:

Initially, determine the amount of SYN3 that will be charged to the batch according to the following formula:
g SYN3/Liter=24.0 g/Liter×[100/(% SYN3 Batch Purity)]

Next, determine the volume of Water for Injection to be charged to the batch according to the following formula:
Volume of Water for Injection (Liters)=Batch Volume (Liters)×0.5

Charge the volume of Water for Injection into a tared compounding vessel equipped with an agitator. Charge and dissolve, with agitation, the Hydroxypropyl-β-cyclodextrin. Note, complete dissolution of Hydroxypropyl-β-cyclodextrin may require approximately 30 to 45 minutes of continuous agitation at a moderate stirring speed. Water for Injection may be used to rinse weighing vessel to retrieve all of the material. Charge and dissolve the Polysorbate 80 to the solution. The Polysorbate 80 may be predissolved in 0.1× total batch volume of Water for Injection (Liters) and charged to the solution.

Charge and dissolve with agitation the SYN3 into the solution. Complete dissolution of SYN3 may require up to 1 hour of mixing. Water for Injection may be used to rinse weighing vessel to retrieve all of the material.

Charge and dissolve with agitation and in order: Ascorbic Acid, Citric Acid monohydrate and Sodium Citrate dihydrate into the solution. Water for Injection may be used to rinse weighing vessels to retrieve all of the material. Add the Water for Injection to bring the batch to the final volume (density of solution is 1.058 g/ml at 25° C.). Mix the solution for a minimum of 15 minutes.

Remove a small (<5 ml) sample of the solution for pH measurement. The pH should be between 5.0 and 6.0. No pH adjustment is necessary. Aseptically filter the solution that has been washed and tested for integrity into a sterilized, stainless steel pressure vessel or equivalent should be used. If necessary, the compounded batch may be stored at 2° C. to 8° C. for up to 24 hours in a sealed, sterilized, stainless steel pressure vessel prior to filling. The batch may be filtered more than once to assure sterility.

Aseptically fill 5.3±0.1 ml of solution into 20-ml Type 1 flint glass vials that have been washed and sterilized. Aseptically insert lyo-shape rubber that have been washed, siliconized and sterilized into the vials in the lyophilization position.

To lyophilize, precool the lyophilizer shelves to 4±2° C. Aseptically load the trays of filled vials onto the lyophilizer shelves. After all the trays are loaded, cool the shelves to −40° C. in 1 hour and maintain the product at −35° C. or below for at least 4 hours before proceeding. Start cooling the condenser. When the condenser temperature is at −45° C. or below, begin evacuating the chamber. When 50-70 mm Hg of vacuum pressure is attained, heat the shelf temperature to −20° C. over 0.5 hour. Maintain the shelf temperature at −20° C. for 36 hours at approximately 150 mm Hg pressure (100 to 200 mm Hg pressure). Product temperature must remain at or above −20° C. for at least 6 hours before proceeding. Heat the shelf to 25° C. in 1 hour and reduce pressure to approximately 50 mm Hg pressure. Maintain the shelf temperature at 25° C. at approximately 50 mm Hg pressure for 14 hours. Vent the chamber with sterile filtered nitrogen to approximately 950 mm Hg. Stopper the vials inside the lyophilizer. Remove the vials from the lyophilizer and crimp the vials with 20-mm aluminum. The vials should be stored at 2° C. to 8° C. until inspection is completed.

The product is a white to off-white cake. The vials should be stored between 2° C. to 8° C. after inspection. For labeling and inspection purposes, the vials may be exposed to 19° C.-25° C. for up to 6 hours.

Recombinant adenoviral vectors (e.g.,p53 (rAD/p53)) can remain stable when combined with the lyophilized formulations of SYN3 for at least 2 hours at 37° C. and 24 hours at 25° C. p53 remains stable when combined with the aqueous solution formulations of SYN3 for at least 4 hours at 37° C. and 24 hours at 25° C.

### Example 7

Synthesis of Compound A-LB

In the following examples, “g” means grams, “ml” means milliliters, “mol” means moles, “° C.” means degrees Centigrade, “min.” means minutes, “DMF” means dimethylformamide, and “PN” specifies particle number. All temperatures are in degrees Centigrade unless otherwise specified.

The following relates the methodology utilized in the synthesis of compound VII, also known as A-LB. Provided below are the synthetic details for compound 5-6 and the steps required for purification.

Materials and Reagents Used
N-(3-aminopropyl)-1,3-diaminepropane
cholic acid
dicyclohexylcarbodiimide (DCC)
isobutyl chloroformate
triethylamine
lactobionic acid

### Experimental

### Synthesis of Compound A-LB

The following relates the methodology utilized in the synthesis of compound VII, also known as A-LB. Provided below are the synthetic details for compound 5-6 and the steps required for purification.

Materials and Reagents Used
N-(3-aminopropyl)-1,3-diaminepropane
cholic acid
dicyclohexylcarbodiimide (DCC)
isobutyl chloroformate
triethylamine
lactobionic acid

Compound 5: A solution of lactobionic acid (716 mg, 2 mmol) in methanol (60 mL) was heated to reflux. To this solution was added DCC (500 mg, 2.5 mmol) and the resultant solution was stirred at reflux. After 2 h, N-(3-aminopropyl)-1,3-diaminepropane (800 mL, 5.7 mmol) was added and the resultant solution was stirred for 1 additional hour. The reaction solution was cooled to room temperature and concentrated to obtain the crude product 5. Crude amide 5 was purified upon trituration with dichloromethane to provide 5 (2.72 g, 5.7 mmol) as a sticky hygroscopic solid.

Compound 6: A solution of cholic acid (4.1 g, 10 mmol)) in DMF (60 mL)was cooled to 0° C. To this solution was added isobutyl chloroformate (1.2 mL, 10.2 mmol) and triethylamine (1.4 mL, 10.4 mmol) and the resultant solution was stirred for 10 minutes followed by addition of 5 (2.5 g, 5.3 mmol) in DMF (40 mL). The reaction solution was stirred for 72 h then concentrated to provide the crude product 6. Crude product was purified by column chromatography to provide the pure 6 (A-LB).

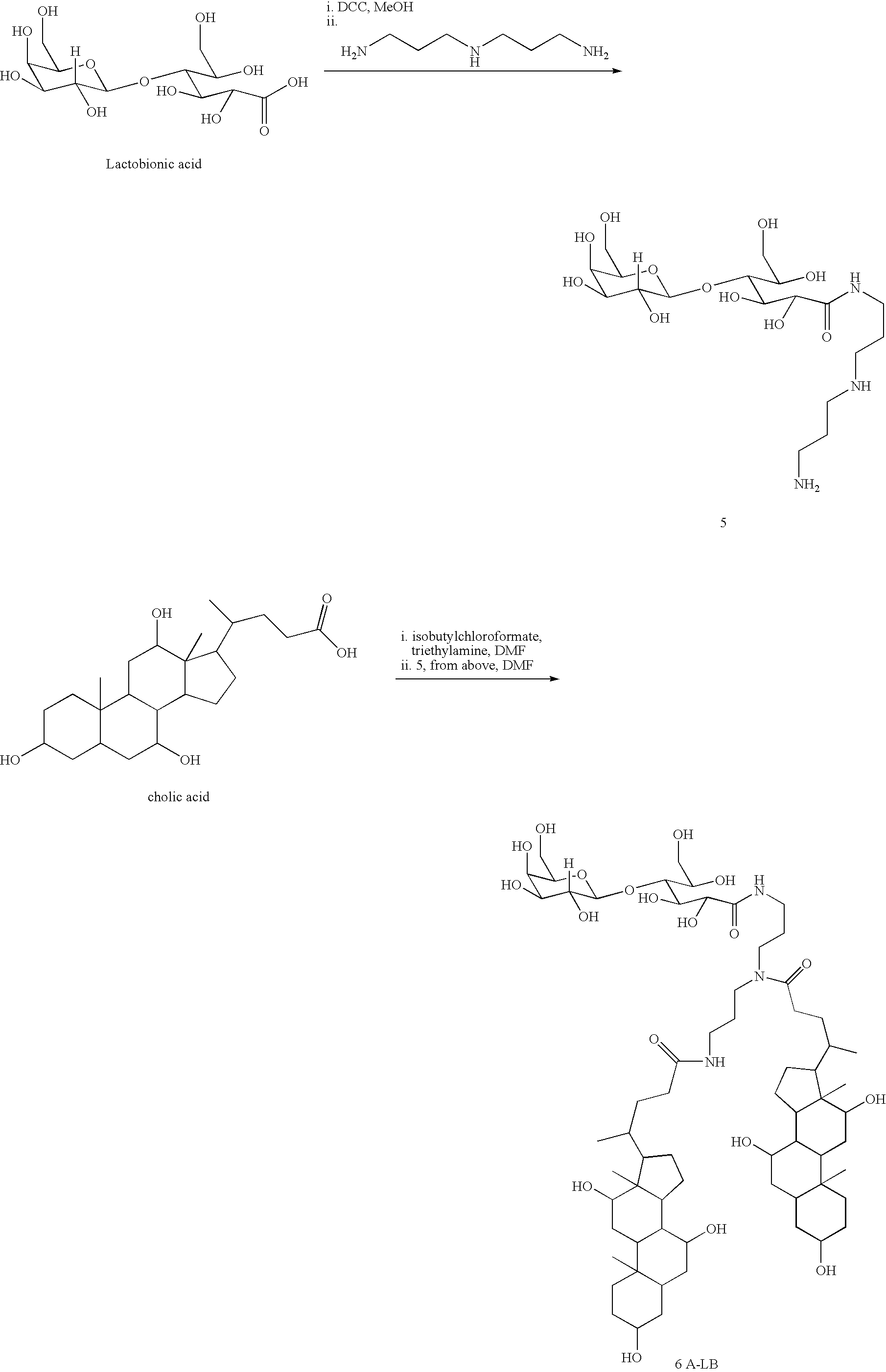

### Example 8

Interferon Therapy in Bladder Cancer.

In this example the use of an adenoviral vector containing the human interferon alpha transgene was evaluated in pre-clinical animal models. Animals that received intravesical administration of rAd-IFN in a SYN3 formulation had very high local concentrations of interferon protein in the urine of animals, with minimal systemic levels of interferon. We have also tested the efficacy of rAd-IFN/SYN3 in an orthotopic tumor model for superficial bladder cancer using athymic mice. Mice that received rAd-IFN/SYN3 had dramatic reduction in the size of bladder tumors compared to controls. Based upon these results, rAd-IFN/SYN3 may represent a new alternative treatment for superficial bladder cancer.

### Example 8A

Antitumor Activity of rAd-IFN

E1-region deleted adenoviruses were constructed that encode human interferon (IFN) (rAd-IFN). For in vivo intravesical delivery studies, we utilized an adenovirus vector encoding the human α2b interferon gene (IACB). Transduction of cells with this vector results in the production of the human α2b interferon protein with primary amino acid sequence identical to INTRON A. The protein contains a 23 amino acid secretory leader peptide linked to a 165 amino acid interferon protein. N-terminal amino acid sequencing has confirmed that the secretory leader peptide is properly cleaved during post-translational modification. In contrast to E. Coli produced recombinant INTRON A, the interferon protein produced form rAd-IFN transduction is glycosylated. The apparent glycosylation of IFN did not affect the detection of rAd-IFN mediated expression of IFN by immunoassay or antiviral bioassay. Bioanalytical methods were developed for the detection of IFN protein from both serum and urine using an electrochemiluminescent immunoassay.

Interferon α species generally display a high level of species specificity in their biological properties. To evaluate the antitumor efficacy of interferon alpha in a mouse tumor model that contains human tumor xenografts, it would be optimal to have an interferon α species capable of binding both the mouse and the human interferon receptors. For in vivo efficacy experiments using a mouse tumor model, we utilized an adenovirus vector that contains a hybrid form of interferon (αb 2/α1). The hybrid interferon protein has anti-tumor activity against both human tumor cells as well as mouse tumor cells (Rehberg et al., J Biol Chem., 257(19):11497-502 (1982)). Since the hybrid interferon is active on mouse cells as well as human cells (Rehberg et al., J Biol Chem., 257(19):11497-502 (1982)), the conformation of the hybrid interferon must be capable of binding and activating the mouse IFN receptor. Therefore, it was believed that the hybrid IFN protein would more closely model the antitumor efficacy of interferon α2b in humans.

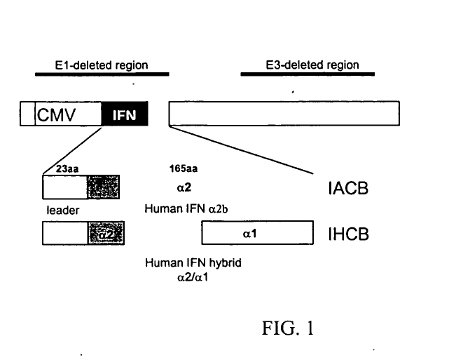

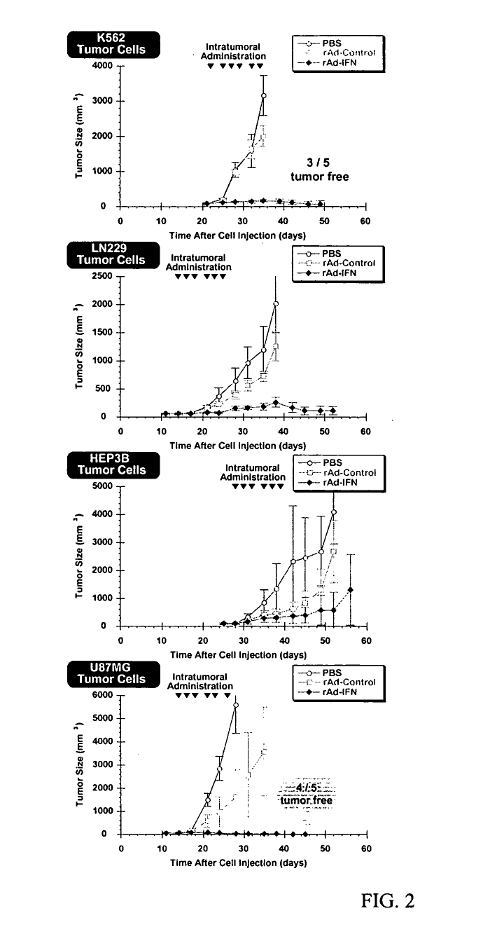

The interferon transgenes are incorporated into the same adenoviral backbone as the vector described as A/C/N 53 in U.S. Pat. No. 6,210,939 incorporated by reference. A schematic representation of the vectors is illustrated in FIG. 1. Following the demonstration of IFN expression and biologic activity of IFN in cell culture and in animals by rAd-IFN, the antitumor efficacy of rAd-IFN in subcutaneous xenograft tumor models was evaluated (FIG. 2). Inhibition of tumor growth was observed following intratumoral rAd-IFN administration in nude mice bearing tumors derived from glioblastoma (LN229, U87MG), hepatoma (HEP3B) and CML (K562). In these studies tumors were established for 10-30 days depending on the tumor model before treatment was initiated.

In the K562 model 3 of 5 mice were tumor free and in the U87MG model 4 of 5 mice were tumor free. Based upon these results, it is clear that rAd-IFN has a potent antitumor activity in subcutaneous solid tumor models in a variety of tumor subtypes. See FIG. 2.

### Example 8B

### SYN3 Eenhancement of Adenovirus Gene Expression

In this example, intravesical administration of recombinant adenovirus can be applied locally into the bladder lumen with only minor systemic exposure. However, single intravesical administration of a human recombinant replication-deficient adenovirus (rAd) has shown only limited gene transfer and expression in previous studies (Bass et al., Cancer Gene Ther., 2(2):97-104 (1995)). It is believed that the structure of the bladder may be responsible for its inability to be transduced by viral vectors. The bladder must act as the primary natural defense against pathogenic bacteria and viruses, while also functioning as a reservoir for containment of urine. The luminal bladder epithelium is covered with a hydrophilic glycosaminoglycan (GAG) layer that hinders passage of urine from the bladder lumen into the tissue. The GAG layer defends against bacterial adhesion (Parsons CL, World J. Urol., 12(1):38-42 (1994)) comparable to other epithelial protective mechanisms (e.g., the gastro-intestinal tract (gastro-intestinal tract ref)). Efforts have focused on identification of agents that could modify this layer to enhance adenoviral attachment and transduction. While ethanol was first identified as an agent that could increase adenoviral gene transfer (Engler et al., Urology, 53(5):1049-53 (1999)), hemorrhagic cystitis was also noted after administration and this formulation was deemed unsuitable. Additional screening of alternative formulations was undertaken at Canji to identify agents that could enhance adenoviral gene transfer to the bladder without the adverse effects of ethanol. Various detergents (cationic, anionic, zwitterionic and non-ionic) were evaluated for their ability to enhance adenoviral delivery. The non-ionic detergent Big CHAP was found to potently enhanced transduction of the bladder epithelium with adenoviral vectors without cystitis. Big CHAP improved gene transfer in a concentration dependent manner, with maximal transduction and saturation of gene expression was reached with Big CHAP concentration of 30 mg/ml (34 mM).

Further characterization of the enhancement activity of Big CHAP revealed variability among different commercially available preparations, suggesting heterogeneity in the detergent's composition. In fact, some preparations of Big CHAP failed to enhance viral transgene expression altogether. When a bioactive lot of Big CHAP was compared to an inactive lot by thin layer chromatography, at least three additional compounds were present in the bioactive lot. MALDI-MS, ¹H-NMR and ¹³C-NMR structural analysis indicated that the three additional components were most likely byproducts from Big CHAP manufacturing processes. Each impurity was isolated and assayed for its ability to enhance viral transgene expression in the bladder by coadministration with rAd-β-gal Impurity #2 was the most potent compound, but was also extremely insoluble. See U.S. Pat. No. 6,392,069 which is incorporated herein by reference in its entirety.

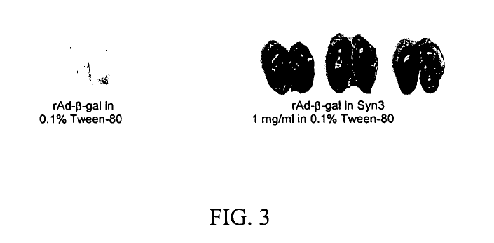

SYN3 was tested for its ability to enhance rAd gene transduction in the bladder epithelium. Rats received intravesical administration of rAd-β-gal (0.5 ml; 7.6×10¹⁰ P/ml) in either a SYN3 formulation or a vehicle formulation (0.1% Tween-80). After 45 minutes, the test article was removed, and the animals allowed to recover. After 2d, the animals were sacrificed, their bladders harvested and X-gal stained to determine the extent of rAd gene expression. Rats that received rAd-β-gal in a SYN3 formulation had a dramatic enhancement in lacZ gene expression compared to delivery of the same virus in a vehicle formulation (FIG. 3).

### Example 8C.

### The Effect of the Combination Treatment on Interferon Expression in the Urinary Bladder.

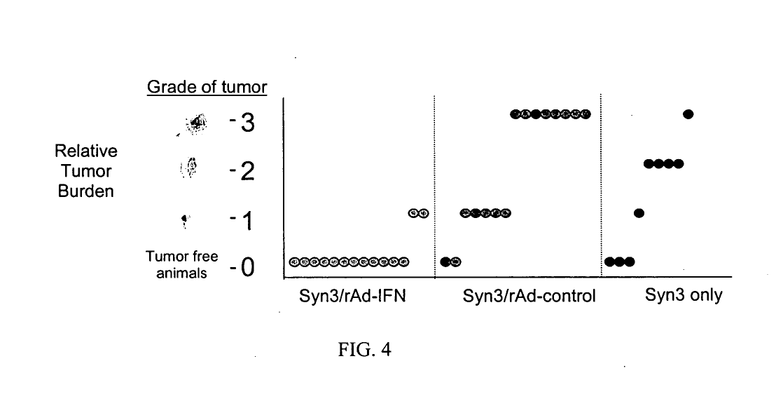

The combination treatment of the urinary bladder with SYN3 with a recombinant adenoviral vector containing the interferon gene on of interferon in the treatment of the urinary bladder provided an unexpectedly enormous increase in the expression of the transgene interferon. Pre-clinical testing demonstrated that SYN3 can dramatically enhance the delivery and expression of a variety of recombinant adenoviral vectors to the bladder epithelium . Studies were performed in mice to quantitate the increase in gene transfer to the bladder epithelium using a SYN3 formulation. Mice received intravesical administration of rAd-IFN in either a SYN3 or vehicle formulation. Two days after treatment, the mice were sacrificed and their bladders harvested and snap frozen on dry ice. Nucleic acids were then extracted from each bladder, and assayed for the level of rAd-IFN DNA and RNA present using PCR and RT-PCR respectively. Mice that received rAd-IFN in a SYN3 formulation had approximately one thousand times greater number of copies of rAd-IFN DNA compared to mice that received rAd-IFN in the vehicle formulation (FIG. 4a), and at least ten thousand times more RNA detected in bladder homogenates than mice that received rAd-IFN in a vehicle formulation

| | DNA | RNA |
|---|---|---|
| | (copies/mg tissue) | (MEQ/mg tissue) |
| rAD-IFN/SYN3 | 1.05e5 | 5.33e7 |
| | 1.44e5 | 1.76e8 |
| | 7.14e5 | 4.00e6 |
| | 1.73e4 | 8.00e6 |
| | 1.71e4 | 5.65e6 |
| Average/SD | 5.81e4/6.2e5 | 4.94e7/7.4e7 |
| rAd-IFN | 3.07e1 | BQL |
| | 1.00e1 | BQL |
| | 2.67e1 | BQL |
| | 3.93e1 | BQL |
| | 2.55e1 | BQL |
| | BQL | BQL |
| Average/SD | 2.37e1/11.6 | BQL |
| SYN3 | BQL | BQL |
| | BQL | BQL |
| | BQL | BQL |
| | BQL | BQL |
| | BQL | BQL |

Table 1. Mice received intravesical administration of rAd-IFN in either a SYN3 formulation (100 μl; 7.4×10¹⁰ P/ml) or a vehicle formulation for 45 minutes. Two 10 days after treatment, the mice were sacrificed and their bladders harvested and snap frozen on dry ice. Nucleic acids were then extracted from each bladder, and assayed for the level of rAd-IFN DNA and RNA present using PCR and RT-PCR respectively. BQL: Below Quantitation Level: DNA: 10 copies/mg tissue; RNA: 1000 MEQ/mg tissue. For comparison the levels obtained by administration of SYN3 alone are also shown. Based upon these results, it is clear that the SYN3 formulation is necessary to enhances the delivery and expression of rAd-IFN in the bladder epithelium.

### Example 8D

### The Efficacy of rAd-IFN/SYN3 for Superficial Bladder Cancer

The efficacy of rAd-IFN/SYN3 was evaluated using an orthotopic tumor model (Watanabe et al., Cancer Gene Ther., 7(12):1575-80 (2000)). Superficial tumors were established in the bladders of athymic mice by intravesical administration of human UMUC-3 transitional carcinoma cells (TCC) following a brief trypsin pre-treatment to enhance tumor cell adhesion. Tumors were allowed to form by growing for a period of 6 days before treatment began. Mice then received intravesical administration of either rAd-IFN/SYN3, rAd-control/SYN3 or SYN3-vehicle alone. Two different control adenovirus constructs were used for comparison in these studies, one containing no transgene (ZZCB) and one containing the human secreted alkaline phosphatase gene (APCB), since rAd-IFN produces a secreted gene product. Mice were administered 100 μl of rAd (1.1×10¹¹ P/ml) in a SYN3 formulation for one hour on two consecutive days. Fourteen days after treatment (20d after initiation of tumors), the mice were sacrificed, their bladders harvested and fixed in formalin, and their tumor burden evaluated by macroscopic examination. Overall percentages of bladders that had tumors were scored, as well as the relative size of the tumors. Tumors were scored on a scale of 0-4 based upon the percentage of the bladder lumen occluded by the tumor. The overall incidence of tumors in the rAd-IFN/SYN3 treated group was significantly lower than the incidence of tumors in the rAd-control or SYN3 only groups (FIG. 4). Notably, the few instances of remaining tumor in the rAd-IFN treated animals had significantly reduced size compared to controls. These results indicate that rAd-IFN/SYN3 greatly reduces the growth of superficial bladder tumors in this orthotopic tumor model.

In other studies, an orthotopic tumor model has been used in which superficial tumors are established in the bladders of athymic mice by intravesical administration of human KU-7 bladder cancer cells stably transfected with the Green Fluorescent Protein (GFP) following trypsin pre-treatment. By surgically exposing and illuminating the bladder with a light to activate GFP fluorescence, the sequential evaluation of tumor size can be monitored in vivo without sacrifice of the animal. Tumor cells were allowed to grow for a period of 10 days, whereupon the bladder was surgically exposed and illuminated, and the fluorescent tumor burden for each animal photographically recorded. Using image analysis software, the percentage area of the bladder containing tumor cells relative to the total area of the bladder was also determined before and after treatment. Mice received intravesical administration of 100 μl of rAd (1×10¹¹ P/ml) for one hour on two consecutive days. Four treatment groups were compared: rAd-IFN/SYN3, rAd-β-gall/SYN3 rAd-IFN alone or SYN3 alone. Twenty-one days after treatment (31d after initiation of tumor growth), the bladders were again inflated, surgically exposed and the GFP tumor burden was again measured as described above.

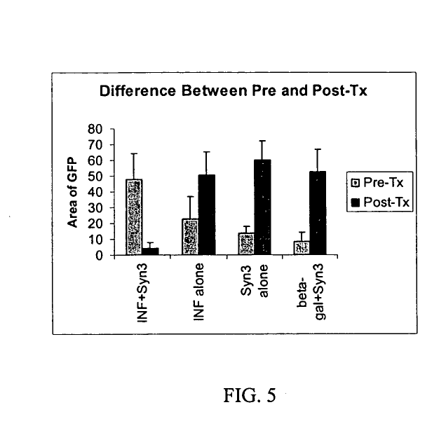

Animals that received the rAd-IFN/SYN3 had a significant decrease in the tumor burden over time (FIG. 5). In contrast, the size of tumors continued to increase in all other treatment groups. The administration of rAd-IFN alone did not cause a significant reduction in tumor growth, indicating the necessity for including the enhancing agent SYN3 in the formulation. Since no reduction in tumor growth is observed in the rAd-β-gal/SYN3 group, the reduction in tumor growth observed in the rAd-IFN/SYN3 group must result as a consequence of interferon transgene expression, and is not merely a non-specific effect due to the administration of the adenovirus vector. In summary, rAd-IFN/SYN3 appears to significantly reduce the growth of superficial bladder tumors in this orthotopic tumor model. To visualize the reduction in tumor burden achieved using the SYN3/rAd-IFN compared to controls, pictures of the tumor burden for individual animals before and after treatment are provided for each treatment groups).

### Example 8E

Detection of rAd-IFN Expression in the Rat Bladder

When cells are transduced with rAd-IFN, a secreted protein product is produced. Therefore, it was hypothesized that the level of viral transgene expression obtained in the bladder epithelium may be estimated by the amount of interferon secreted into the urine. Initial experiments compared the levels of interferon in urine from female Sprague-Dawley rats that received intravesical administration of rAd-IFN or rAd-control (ZZCB) in a SYN3 formulation. Rats received intravesical administration of rAd (5×10¹⁰ P/ml) in SYN3 (1 mg/ml) for 45 minutes and were allowed to recover. Urine was obtained from each animal using a metabolic cage (4 hour collection) 48h post treatment. Rats that received rAd-IFN in the SYN3 formulation had very high levels of interferon protein in their urine (˜25 ng/ml), with only trace levels of interferon in their serum (FIG. 5). In contrast, rats that received rAd-control in a SYN3 formulation had no interferon protein in their urine or serum. After 48h, the rats were sacrificed and their bladders harvested and homogenized to determine the interferon levels. Rats that received SYN3/rAd-IFN had significantly high levels of IFN protein detected in bladder homogenates. No interferon was detected in the bladder homogenates of SYN3/rAd-control animals. These results are consistent with PCR/RT-PCR results and suggest that the urine can be used as a tool to monitor both the level and duration of viral transgene expression in the bladder.

### Example 9

### Part 1: Synthesis of Compound 3

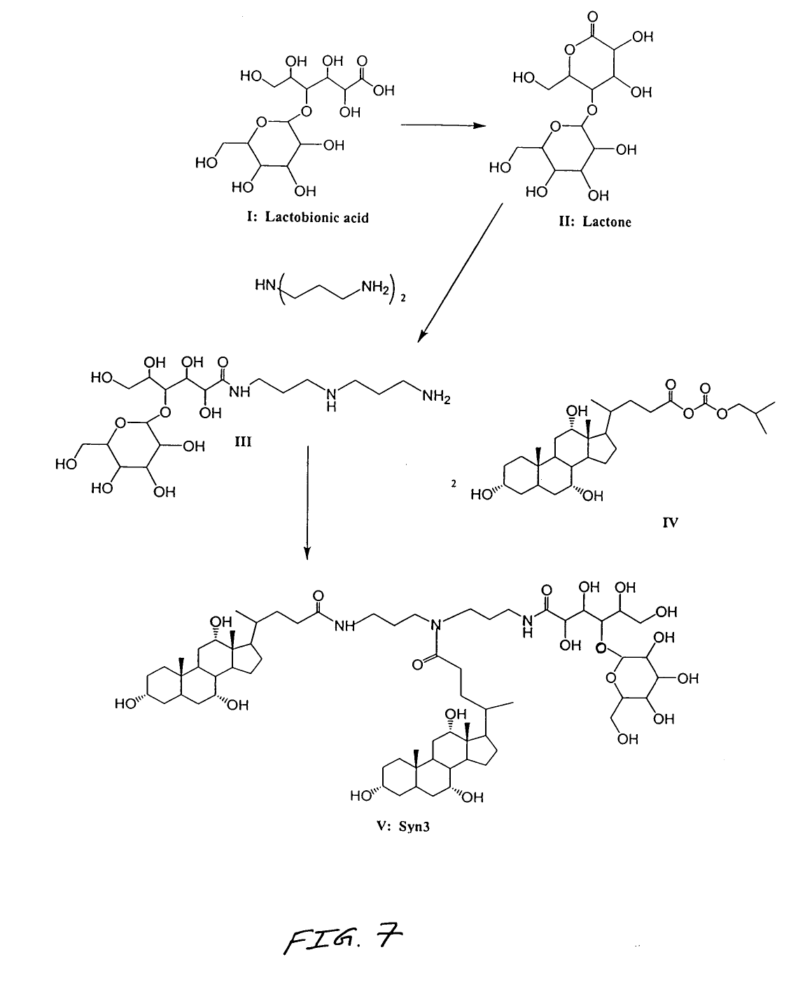

The synthetic scheme for SYN3 is shown in FIG. 7, which is adapted from U.S. Pat. No. 6,392,069. The lactone of lactobionic acid (2) was synthesized by dissolving one g (2.8 mmol) of lactobionic acid (1) in 50 ml of methanol, evaporating to dryness on a rotary evaporator, and repeating this process six times. To obtain Compound 3, the resulting residue (2) was dissolved in 50 ml of isopropanol by heating to 50° C. To this solution was added 1.2 ml (8.4 mmol) of N-3-aminopropyl)-1,2-propanediamene. The temperature was increased to 100° C., and the solution was stirred for three hours. The solvent was removed by rotary evaporation and the resulting residue was washed several times with chloroform to remove excess unreacted N-(3-aminopropyl)-1,3-propanediamene. The remaining residue (3) was used as is in Part 3 below.

### Part 2; Synthesis of Compound 4

Compound 4 was synthesized by dissolving 2.28 g of cholic acid (5.6 mmol) in N,N-dimethylformamide by heating to 60° C. Triethylamine (0.78 ml (5.6 mmol)) was added and the solution was cooled in an ice bath. Isobutyl chloroformate (0.73 ml (5.6 mmol)) was then added and a white precipitate formed as the stirring was continued for ten minutes.

### Part 3; Synthesis of SYN3 (Compound 5)

Compound III was dissolved in N, N-dimethylformamide, cooled in an ice bath, and stirred. The suspension resulting from the synthesis of Compound 4 was filtered into the solution containing Compound 3. The resulting solution was stirred at room temperature for 6 hrs. The solvent was removed using high vacuum rotary evaporation and the residue was dissolved in 100 ml of chloroform/methanol (50/50). Twenty-five ml of this solution was purified by silica gel flash chromatography using chloroform/methanol (60/40) as the elution solvent. Analysis of the fractions eluting from the column was conducted by silica gel thin layer chromatography using a mobile phase consisting of chloroform/methanol/water/concentrated ammonium hydroxide (100/80/10/5). The compounds were visualized by charring after spraying with ethanolic sulfuric acid. Fractions containing product were consolidated and repurified using flash chromatography and chloroform/methanol/water/concentrated ammonium hydroxide (100/80/10/5) as the elution solvent. Fractions containing product were consolidated and evaporated to a white powder (300 mg of Compound 5). ¹H-NMR and MALDI mass spectrometric analysis of the product were consistent with the structure shown.

### Example 11

Preferred delivery enhancing compounds are set forth in the following table. Although the table exemplifies compounds that have a cholic acid substituent, one skilled in the art will readily recognize other steroidal substituents could replace cholic acid without compromising the delivery enhancing properties of the compound. Methods of making such compounds are taught in the Provisional Application, filed Jun. 4, 2003 under Townsend and Townsend and Crew LLP Attorney Docket No.: 016930-000830US, assigned to the same assignee as the present application, and herein incorporated by reference in its entirety and with particular reference to the transfection agents and delivery enhancing agents disclosed therein, the methods of making the same and their use.

Summary of Syn3 Analogues

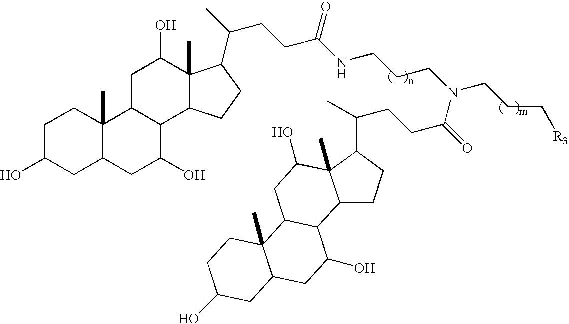

| R-Group | R3 | m, n = |
|---|---|---|
| A-LB Lactobionic acid (Syn3) | 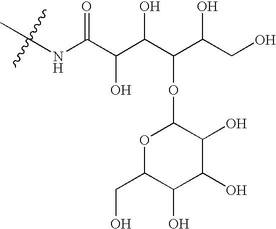 | 1 |
| MB Melibionic acid | 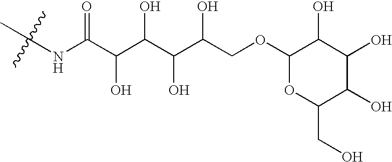 | 1 |
| MT Maltobionic acid | 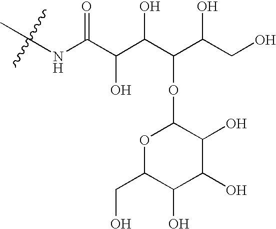 | 1 |
| A-DL Dilactose | 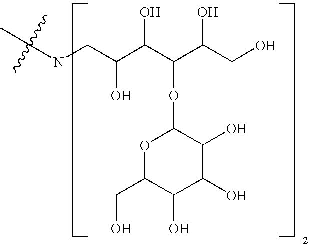 | 1 |
| RLB Reductive aminated lactobionic acid | 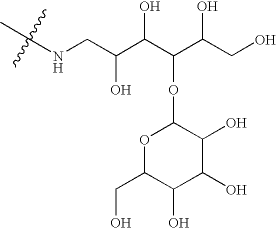 | 1 |
| S-LB Short chain lactobionic acid | 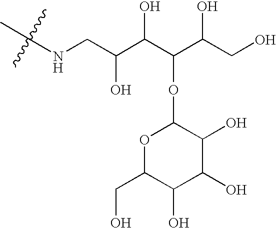 | 0 |
| SC Succinate | 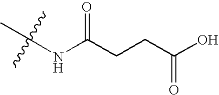 | 1 |
| TMA Trimethylammonium chloride | 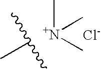 | 1 |
| HCl Hydrochloride | 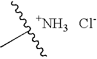 | 1 |
| HOAc Acetate | 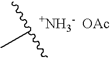 | 1 |
| TEA Triethylammonium chloride | 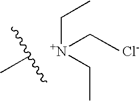 | 1 |
| EDTA Ethylene Diamine Tetraacetic acid | 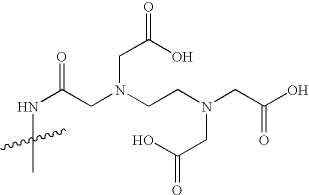 | 1 |
| DA DiAcetic acid | 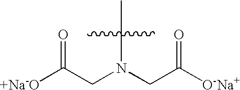 | 1 |
| TMR Tetra-methyl Rhodamine | 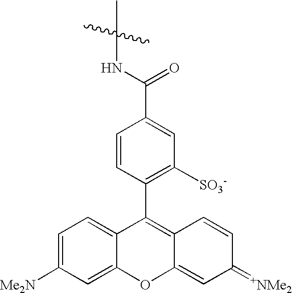 | 1 |
| S-HCl Short chain hydrochloride | 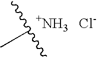 | 0 |

### Example 12

### Uptake of IFN Protein after Intravesical Administration in a SYN3 Formulation

This Example investigated whether SYN3 enhanced the uptake of interferon protein by determining if SYN3 can increase the tissue levels of interferon protein when administered in a SYN3 formulation.

Methods. As the supply of the hybrid IFN protein is limited, the IFNα2b protein (Intron A) was primarily used. For comparison purposes, the hybrid protein was also be included at the time t=0 time point. Outbred HSD rats were anesthetized using isoflurane. Pretreatment urine was collected. The bladder was trans-urethrally catheterized using a catheter and lubricant. The test article was administered to the bladder, the urethra was tied off with 2.0G suture without removing the catheter. After 45 minutes (0 hour), the test article was removed and the animal allowed to recover in the home cage. Urine samples were obtained from rats immediately before sacrifice. After the urine was collected, the bladders were harvested from the rats on that day. The tissue was frozen and assayed for up-regulation of IFN responsive genes.

| | | | Urine | |
|---|---|---|---|---|
| Group | Animal ID's | Sacrifice time | volumes | Treatment |
| 1 | 888, 889 | 0 h post tx | — | IFN α2b |
| | | | | protein in PBS |
| 2 | 876, 877 | 4 h post tx | 876: 1.8 ml | IFN α2b |
| | | | 877: 2.3 ml | protein in PBS |
| 3 | 882, 883 | 1 d post tx | 882: 2.8 ml | IFN α2b |
| | | | 883: 2.9 ml | protein in PBS |
| 4 | 890, 891, 892 | 0 h post tx | — | IFN α2b |
| | | | | protein |
| | | | | in SYN3: |
| | | | | 1 mg/ml |
| 5 | 878, 879 | 4 h post tx | 878: 1.3 ml | IFN α2b |
| | | | 879: 1.2 ml | protein |
| | | | | in SYN3: |
| | | | | 1 mg/ml |
| 6 | 884, 885 | 1 d post tx | 884: 0.8 ml | IFN α2b |
| | | | 885: 0.8 ml | protein |
| | | | | in SYN3: |
| | | | | 1 mg/ml |
| 7 | 880, 881 | 4 h post tx | 880: 1.9 ml | IACB: 1 × |
| | | | 881: 2.2 ml | 10¹¹ P/ml |
| | | | | in SYN3: |
| | | | | 1 mg/ml |
| 8 | 886, 887 | 1 d post tx | 886: 0.8 ml | IACB: 1 × |
| | | | 887: 1.5 ml | 10¹¹ P/ml |
| | | | | in SYN3: |
| | | | | 1 mg/ml |

| Group | Animal ID's | Sacrifice time | Treatment |
|---|---|---|---|
| 1 | 893, 894 | 0 h post tx | IFN α2α1 |
| | | | protein in PBS |
| 4 | 895, 896 | 0 h post tx | IFN α2α1 protein |
| | | | in SYN3: 1 mg/ml |

Materials:

38 Female Harlan Sprague-Dawley rats;

| IACB: | Tris-glycerol formulation | 7.57 × 10¹¹ P/ml |
|---|---|---|
| IHCB: | vPBS formulation | 1.10 × 10¹² P/ml |
| SYN3: | | 6x stock (6 mg/ml) |
| Intron A: | Reference vial used: hydrated in 1 ml of sterile | |
| | nanopure dH2O (10 MIU/ml) | |
| | 950 μl of Intron A diluted with | |
| | 3,008 μl of PBS (2.4 MIU/ml) | |
| | 625 μl of diluted Intron A added to | |
| | 125 μl of either PBS or SYN3 (6 mg/ml) | |
| IFN α2α1 protein: | | 105 μg/ml = |
| | | 105 × 10⁶ pg/ml |
| | | 1.34 × 10⁷ IU/ml |
| | | 1.28 × 10⁸ IU/mg |

IACB is a recombinant adenoviral vector for interferonα2b and has a CMV promoter and a E1-region deletion. IHCB is a recombinant adenoviral vector for hybrid interferon α2α1 also having a CMV promoter and a E1-region deletion.

Preparation of Test Articles:

Prepare IFNα2b at 2.4 MIU/ml final concentration (hydrate one reference vial in 1 ml sterile

Water For Injection)
950 μl Intron A concentrate
3,008 μl PBS
   To prepare the IFNα2b in PBS:
625 μl Intron A @ 2.4 MIU/ml
125 μl PBS
   To prepare the IFNα2b in SYN3:
625 μl Intron A @ 2.4 MIU/ml
125 μl SYN3
   To prepare the rAd-IFNα2b (IACB) in SYN3:
66 μl IACB
250 μl SYN3
1184 μl Tris-glycerol buffer
   Prepare concentrate of IFNα2α1 protein (2.2 ml):
243 μl IFN α2α1 protein
1957 μl PBS
   IFNα2α1/PBS:
625 μl IFNα2α1 protein
125 μl PBS
   IFNα2α1/SYN3:
625 μl IFNα2α1 protein

125 μl SYN3

| | Amount |
|---|---|
| Ingredient | (mg/vial) |
| (SYN3) | 120 |
| Citric Acid Monohydrate USP/EP/Extra Pure or USP | 1.6 |
| Sodium Citrate Dihydrate USP/EP/Extra Pure or USP | 5.1 |
| Hydroxypropyl-β-cyclodextrin (High degree of substitution) | 1000 |
| Polysorbate 80 USP | 60 |

Final concentrations of the excipients were therefore:

| | |
|---|---|
| SYN3: | (120 mg/20 ml)/6 = 1 mg/ml |
| Citric Acid Monohydrate | (1.6 mg/20 ml)/6 = 0.01333 mg/ml |
| Sodium Citrate Dihydrate | (5.1 mg/20 ml)/6 = 0.0425 mg/ml |
| Hydoxy-cyclodextrin | (1000 mg/20 ml)/6 = 8.33 mg/ml |
| Polysorbate 80 (Tween-80) | (60 mg/20 ml)/6 = 0.5 mg/ml |

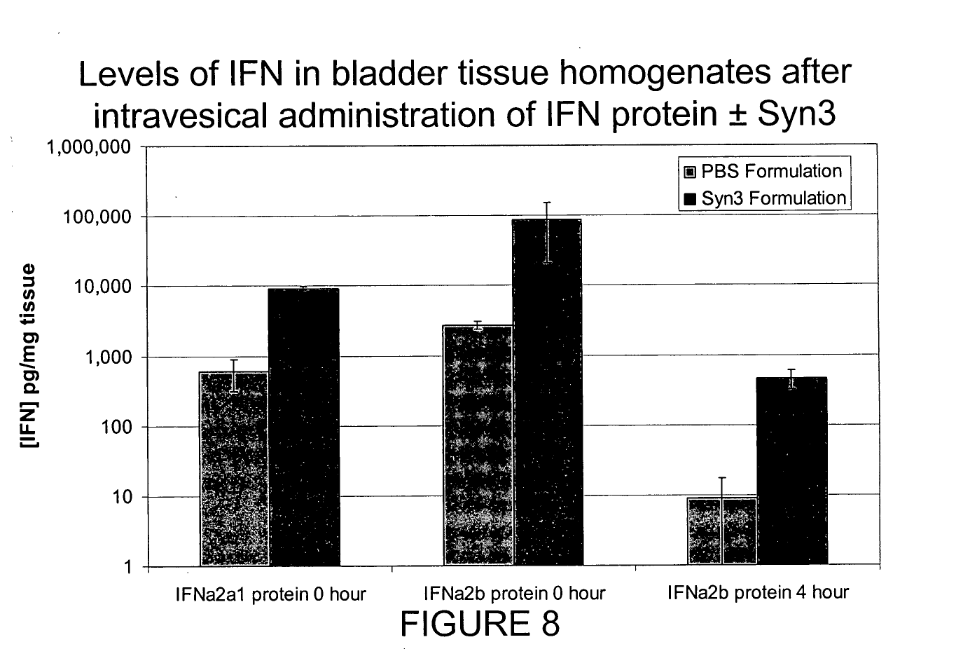

The amounts of IFNα2b present in tissue homogenates was determined using an ELISA assay (PBL). The concentration of protein was measured using a Bradford protein assay. The levels of IFN present in the tissue was expressed as pg IFN/mg tissue (see FIG. 8). As shown in FIG. 8, delivery of IFNα2b in a SYN3 formulation resulted in approximately a 15-fold increase in the amount of detectable IFNα2b protein up to 24 hours after treatment. The delivery of the hybrid IFN protein (IFNα2α1) was also enhanced by delivery in the SYN3 formulation, and was detected at similar levels tissue concentrations as the IFNα2b protein.

### Example 13

### Analysis of Interferon Biological Effects on Bladder Urothelium after Administration of IFN Protein in a SYN3 Formulation

This example investigated if the increase in IFN tissue concentrations resulted in measurable biological responses. To assess biological activity, we used RT-PCR to monitor the expression of IFN responsive genes in rat bladder homogenates after treatment with IFN protein (both Intron A and the ‘universal’ interferon (IFN A/D; IFNα2α1). The following rat genes were assayed: 2′,5′-oligoadenylate synthetase (2′,5′-OAS); the gene encoding the interferon-induced p78 protein (MxAMX1) (MX1); Interferon Regulatory Factor 1 (IRF-1); and Interferonγ IFNγ. (IFNγ is not normally considered an IFN response gene, but is usually expressed after exposure to pathogens such as BCG and was included to determine if a similar IFNγ response would be induced by recombinant adenoviruses). The methods were generally described as above in Example 12. After 1 hour, the test articles were removed and the animals allowed to recover in the home cage. Rats were sacrificed at the times indicated (0 hour=immediately after the treatment) and the samples were snap-frozen in liquid N₂ and submitted for RT-PCR analysis.

| Group | Animal ID's | Sacrifice time | Treatment |
|---|---|---|---|
| 1 | 193, 194 | 0 h post tx | IFN α2b protein in PBS |
| 2 | 176, 177 | 4 h post tx | IFN α2b protein in PBS |
| 3 | 185, 186 | 1 d post tx | IFN α2b protein in PBS |
| 4 | 190, 191, 192 | 0 h post tx | IFN α2b protein |
| | | | in SYN3: 1 mg/ml |
| 5 | 178, 179, 180 | 4 h post tx | IFN α2b protein |
| | | | in SYN3: 1 mg/ml |
| 6 | 187, 188, 189 | 1 d post tx | IFN α2b protein |
| | | | in SYN3: 1 mg/ml |
| 7 | 181, 182 | 4 h post tx | IACB: 1 × 10¹¹ P/ml |
| | | | in SYN3: 1 mg/ml |
| 8 | 183, 184 | 1 d post tx | IACB: 1 × 10¹¹ P/ml |
| | | | in SYN3: 1 mg/ml |
| 1 | 293, 294 | 0 h post tx | IFN α2α1 protein in PBS |
| 2 | 276, 277 | 4 h post tx | IFN α2α1 protein in PBS |
| 3 | 285, 286 | 1 d post tx | IFN α2α1 protein in PBS |
| 4 | 290, 291, 292 | 0 h post tx | IFN α2α1 protein |
| | | | in SYN3: 1 mg/ml |
| 5 | 278, 279, 280 | 4 h post tx | IFN α2α1 protein |
| | | | in SYN3: 1 mg/ml |
| 6 | 287, 288, 289 | 1 d post tx | IFN α2α1 protein |
| | | | in SYN3: 1 mg/ml |
| 7 | 281, 282 | 4 h post tx | IHCB: 1 × 10¹¹ P/ml |
| | | | in SYN3: 1 mg/ml |
| 8 | 283, 284 | 1 d post tx | IHCB: 1 × 10¹¹ P/ml |
| | | | in SYN3: 1 mg/ml |

Materials Needed:

38 female Harlan Sprague-Dawley rats; transferred from protocol 04-634

| | | |
|---|---|---|
| IACB: | Tris-glycerol formulation | 7.57 × 10¹¹ P/ml |
| IHCB: | vPBS formulation | 1.10 × 10¹² P/ml |
| SYN3 | | 6x stock (6 mg/ml) |
| D-PBS | | |
| Tris-glycerol | | |
| buffer: | | |
| Sterile WFI | | |
| IFN α2α1 | | 105 μg/ml = 105 × 10⁶ pg/ml |
| protein: | | |
| | | 1.34 × 10⁷ IU/ml |
| | | 1.28 × 10⁸ IU/mg |

Preparation of Test Articles:
1) IFNα2b/PBS: 5 ml @ 2 MIU/ml Final Concentration

Hydrate one reference vial (10 MU/vial) in 1 ml sterile water for injection 1,000 μl Intron A concentrate
4,000 μl PBS
   2) IFN α2b/SYN3: 10 ml @ 2 MIU/ml Final Concentration

Hydrate two reference vials with 2 ml sterile water for injection 2,000 μl Intron A concentrate
6,334 μl PBS
1,666 μl SYN3
   IACB/SYN3: 4.5 ml @ 1.0×10¹¹ P/ml in SYN3
660 μl IACB
750 μl SYN3
3,090 μl Tris-glycerol buffer
   IFNα2α/PBS: 4 ml @ 1 MIU/ml final concentration
298 μul IFNα2α1 protein
3,702 μl PBS
   IFNα2α1/SYN3: 6 ml @ 1 MIU/ml final concentration
444 μl IFNα2α1 protein
4,556 μl PBS
1,000 μl SYN3
   IHCB/SYN3 4.0 ml @ 1.0×10¹¹ P/ml in SYN3
364 μl IHCB
667 μl SYN3
2,969 μl Tris-glycerol buffer

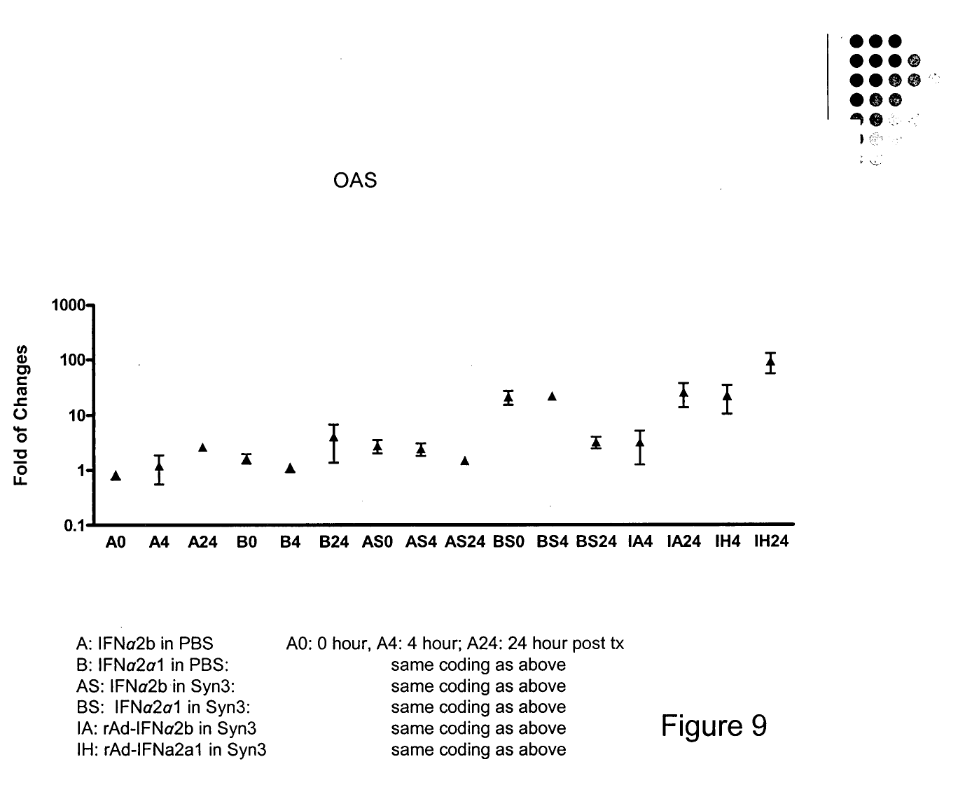

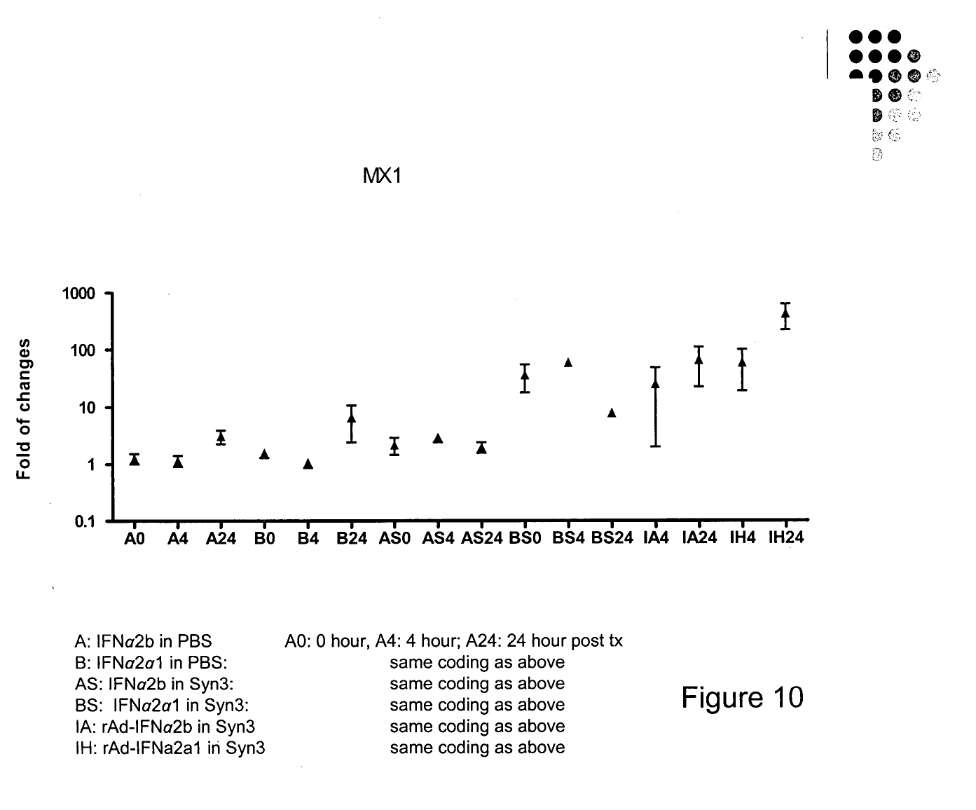

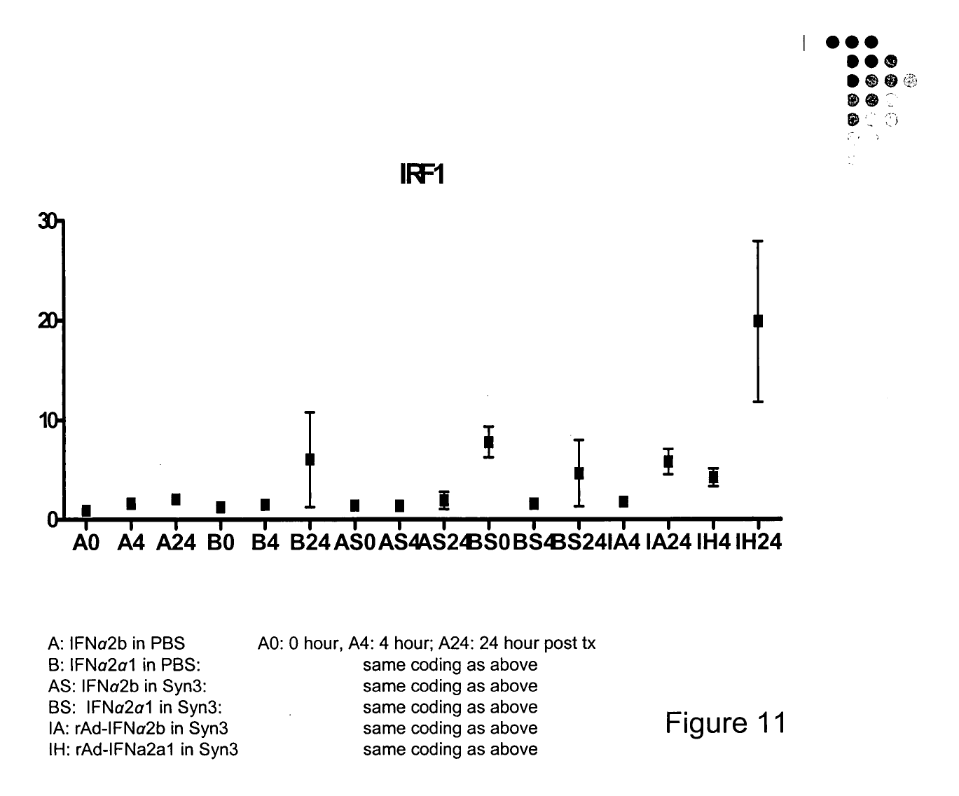

The animals were sacrificed at the indicated times (see FIGS. 9-11) and their bladders harvested on liquid nitrogen for RT-PCR analysis. The primary analysis was to compare the levels of mRNA for the above genes to the level that is observed after Intron A/PBS delivery. Therefore, in the graphs provided, the level of gene activation is normalized to the Intron A/PBS group (1.0).

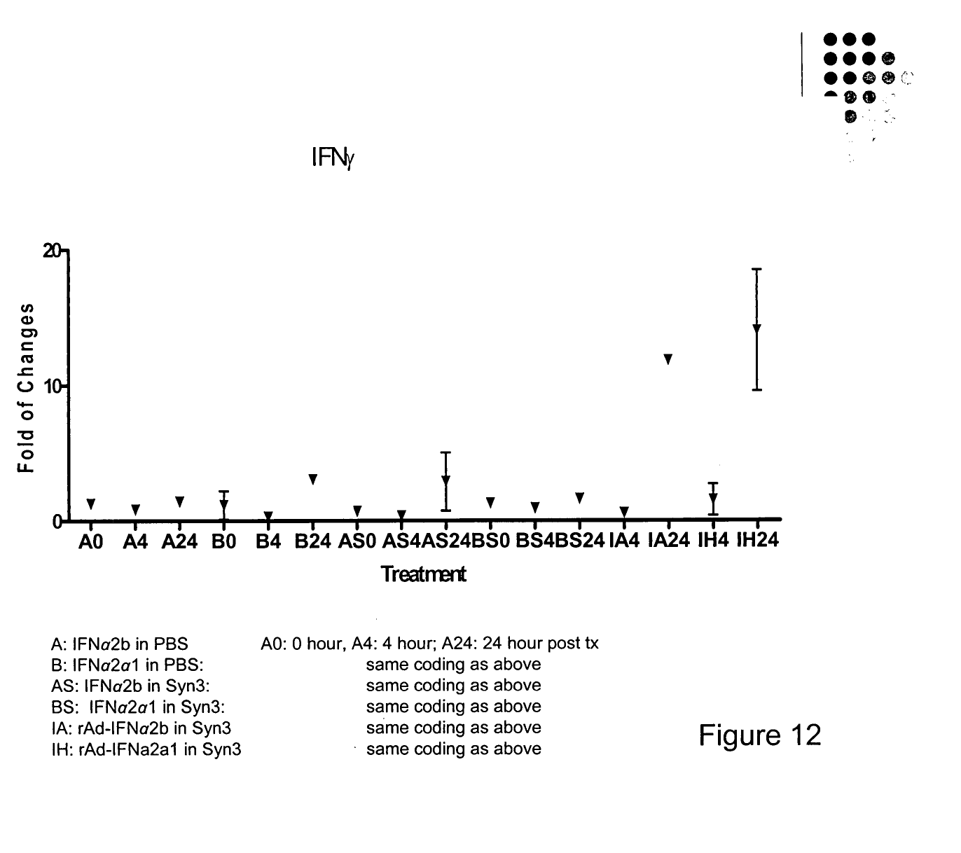

As shown in FIGS. 9-12, the addition of SYN3 increased the expression of known downstream IFN-activated genes (2′-5′OAS, MX1) compared to delivery of the same amount of protein in a PBS formulation. The hybrid protein in SYN3 (BS: IFNα2α1/SYN3) appeared to provide more potent biological responses even though it was dosed at 1 MfU/ml instead of the 2MIU/ml for the IFNα2b. While both Intron A (IFNα2b) and the hybrid IFN (IFNα2α1) increased the expression of the rat OAS and MX1 genes when administered in a SYN3 formulation, both were somewhat inferior to the levels obtained after administration of rAd-IFNα2b or rAd-IFNα2α1.

It is understood that the examples and embodiments described herein are for illustrative purposes only and that various modifications or changes in light thereof will be suggested to persons skilled in the art and are to be included within the spirit and purview of this application and scope of the appended claims. All publications and patent applications cited in this specification are herein incorporated by reference in their entirety for all purposes as if each individual publication or patent application were specifically and individually indicated to be incorporated by reference.

1. A pharmaceutical composition comprising 1) a delivery enhancing agent selected from the group consisting of SYN3 and SYN3 homologs and 2) an interferon protein or an interferon gene delivery system, wherein the gene delivery system contains a gene for the interferon and wherein the gene for the interferon is operably linked to a genetic regulatory element which modulates expression of the gene.

2. The pharmaceutical composition of claim 1, wherein the composition comprises a pharmaceutically acceptable carrier.

3. The pharmaceutical composition of claim 1, wherein the composition is lyophilized.

4. The pharmaceutical composition of claim 1, wherein the composition comprises SYN3.

5. The pharmaceutical composition of claim 1, wherein the interferon is selected from the group consisting of α-interferon, β-interferon interferon, δ-interferon, γ-interferon, and a fusion interferon thereof.

6. The pharmaceutical composition of claim 1, wherein the interferon is selected from the group consisting of interferon α-2b, a fusion interferon α-/2α-1, and interferon α-2e.

7. The pharmaceutical composition of claim 1, wherein the agent is SYN3, and the interferon is human α interferon or a fusion interferon thereof.

8. The pharmaceutical composition of claim 7, wherein the α interferon is an α₁ interferon or an α2 interferon.

9. The pharmaceutical composition of claim 1, wherein the interferon is human interferon.

10. The pharmaceutical composition of claim 1, wherein the homolog is a compound of the following formula: 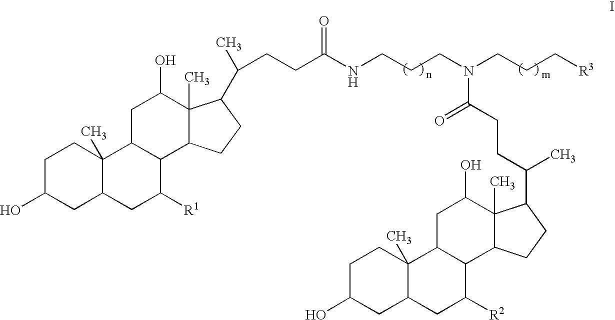
wherein:
R¹ and R² are each independently a member selected from the group consisting of hydrogen, and a hydroxyl group;
m and n are each independently selected from about 0-2;
R³ is selected from the group consisting of —NR⁴R⁵ wherein R⁴ and R⁵ are each independently a member selected from the group consisting of a hydrogen, a saccharide residue, an optionally substituted alkyl, an optionally substituted acyl, and an optionally substituted acyloxy, and a quaternary ammonium salt —NR⁶R⁷R⁸X wherein R⁶, R⁷ and R⁸ are independently a member selected from the group consisting of hydrogen and C₁-C₄ alkyl, and X is the negatively charged ionically bound counterion selected from the group consisting of halogen and an optionally substituted carboxylate.

11. The composition of claim 10, wherein R¹ and R² are both hydroxyl groups.

12. The composition of 10, wherein m and n are each 1.

13. The composition of claim 12, wherein said compound has Formula II:

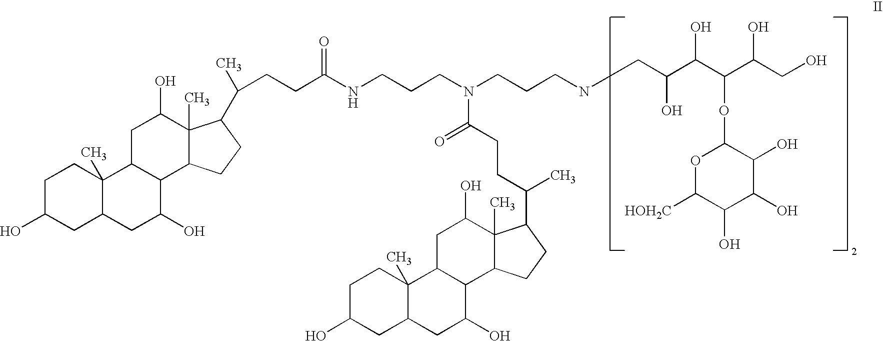

14. The composition of claim 10, wherein R⁴ is hydrogen; and R⁵ is a member selected from the group consisting of a hydrogen, a saccharide residue, an optionally substituted alkyl, an optionally substituted acyl, and an optionally substituted acyloxy.

15. The composition of claim 10, wherein said compound has Formula III:

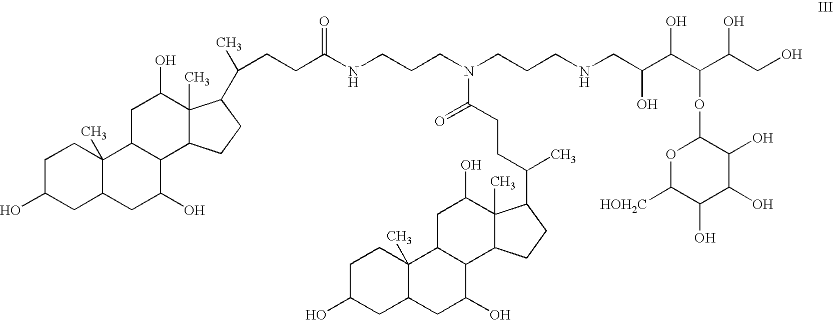

16. The compound of claim 10, wherein R⁴ is succinyl.

17. The compound of claim 10, wherein R⁴ is acetyl.

18. The compound of claim 10, wherein R³ is a trimethylammonium salt.

19. The compound of claim 10, wherein R³ is a triethylammonium salt.

20. A method for delivering interferon to a mammalian subject, said method comprising administering to the epithelial tissue or organ of the subject a therapeutically effective amount of a pharmaceutical composition comprising a delivery enhancing agent selected from the group consisting of SYN3 and SYN3 homologues and administering interferon or an interferon gene delivery system, wherein the gene delivery system contains a gene for the interferon and wherein the gene for the interferon is operably linked to a genetic regulatory element which modulates expression of the gene.

21. The method of claim 20, wherein the agent is SYN3.

22. The method of claim 20, wherein the agent is SYN3 and the concentration of the SYN3 in the composition is from 0.1 mg to 10 mg/ml.

23. The method of claim 20, wherein the interferon is selected from the group consisting of α-interferon, β-interferon interferon, δ-interferon, γ-interferon, and a fusion interferon thereof.

24. A method of claim 23, wherein the interferon is selected from the group consisting of interferon α-2b, a fusion interferon is α-/2α-1, and interferon α-2e.

25. The method of claim 23, wherein the subject is human, the agent is SYN3, and the interferon is human α interferon or a fusion interferon thereof.

26. The method of claim 25, wherein the α interferon is an α₁ interferon or an α₂ interferon.

27. The method of claim 20, wherein the interferon is human interferon.

28. The method of claim 23, wherein the composition is administered intravesically to the tissue or organ.

29. The method of claim 23, wherein the tissue or organ is cancerous.

30. The method of claim 29, wherein the tissue or organ is the human urinary bladder.

31. The method of claim 30, wherein the administering is at intervals selected from the group consisting of about once a week, twice a month, monthly, and bi-monthly.

32. The method of claim 30, wherein the administering is modulated with respect to frequency or the amount of the composition administered to maintain the expression of the interferon gene within a predetermined range.

33. The method of claim 30, wherein composition is administered in a volume from about 50 to 600 ml.

34. The method of claim 33, wherein the volume is from about 100 to 300 ml.

35. The method of claim 34, wherein the composition is administered by catheter.

36. The method of claim 35, wherein the catheter is a balloon catheter and the balloon portion of the catheter is inflated after insertion to reduce the void volume of the bladder.

37. The method of claim 36, wherein the subject is a relapsed or recurrent bladder cancer patient.

38. The method of claim 37, wherein the subject was previously treated with BCG therapy.

39. The method of claim 20, wherein the tissue or organ is an airway tissue or organ, a upper or lower gastrointestinal tissue or organ, or a peritioneal tissue or organ.

40. The method of claim 30, wherein the composition is administered over a period from about 5 minutes to about 2 hours.

41. The method of claim 30, wherein the concentration of SYN3 in the composition is in an amount from 0.1 to 10 mg/ml.

42. The method of claim 23, wherein the homolog has a formula: 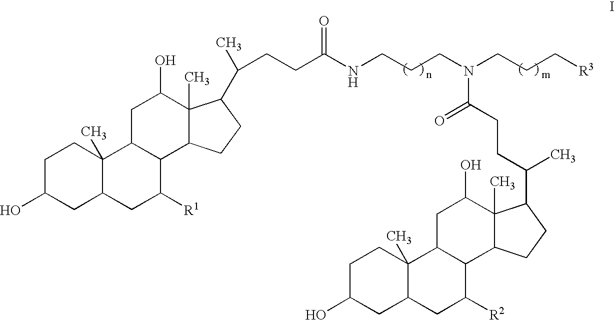
wherein:
R¹ and R² are each independently a member selected from the group consisting of hydrogen, and a hydroxyl group;
m and n are each independently selected from about 0-2;
R³ is selected from the group consisting of —NR⁴R⁵ wherein R⁴ and R⁵ are each independently a member selected from the group consisting of a hydrogen, a saccharide residue, an optionally substituted alkyl, an optionally substituted acyl, and an optionally substituted acyloxy, and a quaternary ammonium salt —NR⁶R⁷R⁸X wherein R⁶, R⁷ and R⁸ are independently a member selected from the group consisting of hydrogen and C₁-C₄ alkyl, and X is the negatively charged ionically bound counterion selected from the group consisting of halogen and an optionally substituted carboxylate.

43. The method of claim 20, wherein the SYN3 or SYN3 homolog is administered in a co-formulation with the interferon or gene delivery system.

44. The method of claim 20, wherein the SYN3 or SYN3 homolog composition and the interferon or interferon gene delivery system are administered separately.

45. The method of claim 44, wherein the SYN3 or SYN3 homolog composition is administered prior to the administration of the interferon or interferon gene delivery system.

46. A kit comprising:
a first container containing SYN3 or a SYN3 homologue capable of enhancing the delivery of an interferon; and
a second container containing the interferon or a gene delivery system, wherein the gene delivery system contains a gene for the interferon and wherein the gene for the interferon is operably linked to a genetic regulatory element which modulates expression of the gene.

47. The kit of claim 46, wherein the first container contains a lyophilized formulation of SYN3.

48. The kit of claim 46, wherein the second container contains a lyophilized formulation of the interferon or the gene delivery system.

49. The kit of claim 46, wherein the interferon gene is an α-interferon gene.

50. The kit of claim 46, wherein the interferon gene is human.

51. A pharmaceutical composition comprising a gene delivery enhancing agent selected from the group consisting of SYN3 and a SYN3 homolog; and a gene delivery system, wherein the gene delivery system contains a gene for interferon and wherein the gene for interferon is operably linked to a genetic regulatory element which modulates expression of the gene.

52. The pharmaceutical composition of claim 51, wherein the gene delivery system comprises a recombinant viral vector.

53. The pharmaceutical composition of claim 52, wherein the recombinant viral vector is selected from the group consisting of a herpes viral vector, retroviral vector, vaccinia viral vector, and an adenoviral vector.

54. The pharmaceutical composition of claim 53, wherein the agent is SYN3, the interferon is human α interferon or a fusion interferon thereof, and the gene delivery system is a recombinant adenoviral gene delivery system.

55. The pharmaceutical composition of claim 51, wherein the composition is in unit dosage format and contains a therapeutic amount of the composition in which the amount of the agent is from 1 to 2000 mg.

56. A method for delivering an interferon to a mammalian subject, said method comprising administering to the epithelial tissue or organ of the subject a therapeutically effective amount of a pharmaceutical composition comprising a delivery enhancing agent selected from the group consisting of SYN3 and SYN3 homologues and a gene delivery system, wherein the gene delivery system contains a gene for the interferon and wherein the gene for the interferon is operably linked to a genetic regulatory element which modulates expression of the gene.

57. The method of claim 51, wherein the gene delivery system comprises DNA and a cationic lipid.

58. The method of claim 51, wherein the urine is monitored to determine expression of the interferon.
